(19) United States
(12) Patent Application Publication
(10) Pub. No.: US 2012/0316203 A1
(43) Pub. Date: Dec. 13, 2012

(54) Compositions and Methods for Inhibition of Cancers

(75) Inventors: Periannan Kuppusamy, New Albany, OH (US); Kalman Hideg, Budapest (HU)

(73) Assignee: THE OHIO STATE UNIVERSITY RESEARCH FOUNDATION, Columbus, OH (US)

(21) Appl. No.: 13/382,545

(22) PCT Filed: Jul. 6, 2010
(86) PCT No.: PCT/US10/41103

§ 371 Date: Apr. 25, 2012

Related U.S. Application Data
(60) Provisional application No. 61/223,283, filed on Jul. 6, 2009.

(51) Int. Cl. A61K 31/454; C12N 5/09; A61P 35/00; C07D 401/06

(52) U.S. Cl. 514/326; 546/208; 435/375

(57) ABSTRACT

Methods and compositions for treating cancers, including ovarian cancers, are described. The compositions generally include a redox based curcumin derivative, diarylidenylpiperiden-4-one (DAP) having a hydroxylamine moiety attached thereto.

### CROSS-REFERENCE TO RELATED APPLICATIONS

This application claims the benefit of U.S. Provisional Application Ser. No. 61/223,283 filed Jul. 6, 2009, the entire disclosure of which is expressly incorporated herein by reference.

### STATEMENT REGARDING FEDERALLY SPONSORED RESEARCH

This invention was made with Government support and the Government has rights in this invention under the grant under the National Institutes of Health Grant No. CA102264 and Hungarian Research Fund Grant OTKA T048334.

### TECHNICAL FIELD AND INDUSTRIAL APPLICABILITY OF THE INVENTION

The present invention relates to compositions and methods for detecting, treating, characterizing, and diagnosing ovarian cancer.

### BACKGROUND

Ovarian cancer is the second most commonly diagnosed gynecological malignancy, and is the leading cause of reproductive cancer mortality among women in the United States. It is estimated that in 2007 there were over 22,430 new cases of ovarian cancer in the United States, resulting in over 15,000 deaths. The current standard of care includes primary surgical cytoreduction followed by cytotoxic chemotherapy; however, recurrence remains a significant problem. Hence, there is a need to develop effective therapeutic agents with minimized untoward side effects.

Curcumin is a beta-diketone constitute of turmeric which is derived from the rhizome of plant Curcuma longa. It has been shown to have antiproliferative, and antiangiogenic activities in a wide variety of tumor cells including inducing cell cycle arrest and apoptosis; inhibiting cell adhesion and angiogenesis. However, its clinical use has been limited due to its low anti-cancer potency and poor absorption.

A class of curcumin analogs, diarylidenylpiperiden-4-one (DAP), has been developed by changing the beta-diketone structure and aryl substitution. These compounds have been shown to have improved anti-cancer activity. For example, DAP-F(o) (EF-24) has been found to have IC50 lower than both cisplatin and curcumin. However, these curcumin analogs are nonspecific cytotoxic compounds that are associated with undesirable side effects by damaging normal cells. For example, many chemotherapy agents act by producing free radicals, which may also cause untoward oxidative stress to normal cells.

There is a need to use antioxidants that can differentiate between “normal” versus “abnormal tumor” cells or tissues in terms of scavenging free radicals.

### SUMMARY

There are provided herein methods of treatment of tumorigenic diseases using a redox based curcumin derivative composition that includes a nitroxide moiety.

There are also provided herein compositions of matter and pharmaceutical compositions, and to methods for their use in the treatment of cancer. For example, a composition of the invention can be a combination of two or more compositions described herein and/or a combination of two or more forms of a composition described herein.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIGS. 1A-1D: Inhibition of cell viability and proliferation by HO-3867. Structures of curcumin, H-4073, and HO-3867 are shown. H-4073 is a 3,5-diarylidenyl piperidone containing a para-fluorosubstitution on the phenyl groups. HO-3867 contains an N-hydroxy-pyrroline moiety covalently linked to the NH₂-terminus of piperidone. In aerated solutions and cells, the N-hydroxy-pyrroline undergoes conversion to and exists in equilibrium with the nitroxide (>NO) form (shown in the circle).

FIG. 1A: Dose-dependent effect of curcumin, H-4073, and HO-3867 on the viability of A2780 cells. Cells were incubated with curcumin, H-4073, or HO-3867 for 24 hours followed by measurement of cell viability (by MTT assay). Columns, mean (n=5); bars, SEM.

FIG. 1B: Dose-dependent effect of H-4073 and HO-3867 on the colony-forming ability of A2780 cells. Columns, mean (n=5); bars, SEM.

FIG. 1C: Effect of H-4073 and HO-3867 (10 μmol/L; 24 h) on the viability of different ovarian cancer cell lines: A2780R (cisplatin-resistant variation of A2780), SKOV3, OV3, and OVCAR3. Columns, mean (n=5); bars, SEM.

FIG. 1D: Dose- and incubation time-dependent effect of HO-3867 (10 μmol/L) on the viability of hOSE, a human ovary surface epithelial cell line used as a healthy control. Columns, mean (n=5); bars, SEM. *, P<0.05 versus the effect of H-4073 at equivalent doses.

FIGS. 2A-2D: Modulation of cell cycle progression and cell cycle regulatory proteins by HO-3867. A2780 cells were treated with HO-3867 for 6, 12, or 24 hours.

FIG. 2A: Representative flow cytometry profiles of control (0 μmol/L) and HO-3867 (20 μmol/L) treatment groups at different time periods.

FIG. 2B: Quantitative cell cycle (DNA content) distribution (% of total) in the control and treatment groups. Columns, mean (n=5); bars, SD; *, P<0.01 versus control.

FIG. 2C: Immunoblot images of cell cycle regulatory proteins.

FIG. 2D: Quantitative results of p53, p21, cdk2, and cyclin A bands. Columns, mean (n=5); bars, SD; *, P<0.01 versus control (0 μmol/L); a.u., arbitrary units.

FIGS. 3A-3B: Induction of apoptosis by HO-3867. A2780 cells were treated with HO-3867 for 24 hours and subjected to Western blot analysis for apoptotic marker proteins.

FIG. 3A: Representative immunoblot images of FAS/CD95, cleaved caspases (cle. casp; 8, 7, and 3), and PARP.

FIG. 3B: Quantitative results of immunoblot. Columns, mean (n=5) expressed as arbitrary units; bars, SD; *, P<0.01 versus control (0 μmol/L).

FIGS. 4A-4C: Inhibition of JAK/STAT3-signaling and downstream proteins by HO-3867. Cells were treated with HO-3867 for 24 hours and subjected to Western blot analysis.

FIG. 4A: Representative immunoblot images of phosphorylated and total STAT3 and JAK1 in A2780 cells and immunoprecipitation results of pSTAT3 (Tyr705/Ser727) and pJAK1 (Tyr1022/1023) using bands captured by STAT3 or JAK1 and blotted with pSTAT3 or pJAK1.

FIG. 4B: Representative Western blots obtained from four other ovarian cancer cell lines (A2780R, SKOV3, OV4, and OVCAR3) treated with 10 μmol/L HO-3867 for 24 hours. Note the decreased levels of pJAK1 and pSTAT3, and corresponding increased levels of cleaved caspase-3 and cleaved PARP in the treated cells compared with nontreated cells. Densitometric analyses of Western blots are shown for pJAK1, pSTAT3, cleaved caspase-3, and cleaved PARP. Columns, mean (n=5) expressed as arbitrary units; bars, SD.

FIG. 4C: Immunoblot images of Bcl-xL, Bcl-2, survivin, cyclin D1, and VEGF proteins in cells. Quantitative results of Bcl-xL, Bcl-2, survivin, and VEGF bands are shown. Columns, mean (n=5) expressed as a percent of control (*, P<0.01); bars, SD.

FIGS. 5A-5D: Effect of HO-3867 on murine xenograft tumors.

FIG. 5A: Dose-dependent decrease in the volume of the xenograft tumors growth is observed following HO-3867 treatment.

FIG. 5B: Final volume of HO-3867-treated tumors at the 5th week.

FIG. 5C: Change in body weight.

FIG. 5D: Consumption of feed containing HO-3867 over time. Points, mean from nine mice in each group; bars, SEM. *, P<0.05 versus nontreated control group.

FIGS. 6A-6D: Effect of HO-3867 on the expression of JAK/STAT3 and targeting genes.

FIG. 6A: Immunoblot analysis using tissue lysates of xenograft tumors. The decreased expression of pSTAT3 Tyr705 and Ser727 and JAK1 are noted in the HO-3867-treated tumor lysates in a dose-dependent manner in concert with decreased expression of both Tyr705-phosphorylated and Ser727-pSTAT3.

FIG. 6B: The decreased expression is also shown in cyclin D1, Bcl-2, and VEGF in a dose-dependent manner.

FIG. 6C: Cleavage of caspase-3 and PARP in HO-3867-treated tumor lysates in a dose-dependent manner.

FIG. 6D: Quantification of cleaved caspase-3 and cleaved PARP. *, P<0.05 versus respective untreated control group.

FIG. 7: Cytotoxicity of DAPs to cancer cells. A number of established human cancer cell lines, namely A2780 (ovarian), A2780R (cisplatin-resistant ovarian), MCF-7 (breast), HCT-116 (colon), PC-3 (prostate), HepG2 (liver), A549 (lung), and SCC4 (squamous cell carcinoma), were exposed to 10 μM DAP H-4073 (FIG. 7A), HO-3867 (FIG. 7B), H-4318 (FIG. 7C), HO-4200 (FIG. 7D)) for 24 h followed by measurement of cell viability by MIT assay. The viable cells were quantified as means±SE (N=6) and expressed as percentage of respective untreated controls. The data show that DAPs induced substantial loss of cell viability in all cancer cell lines tested.

FIGS. 8A-8B: Cytotoxicity of DAPs to noncancerous human cells. Cells were exposed to 10 PM DAPs for 24 h followed by measurement of cell viability by MIT assay. The viable cells were quantified as the mean±SE (N=6) and expressed as percentage of the respective (untreated) controls.

FIG. 8A: Viability of noncancerous cells, namely hOSE (human ovarian surface epithelial), HSMC (human smooth muscle cell), and HAEC (human aortic endothelial cell). The data show that DAPs were cytotoxic to all three noncancerous cell lines tested; however, the cytotoxicity was significantly less in the case of HO-3867 and HO-4200 compared to H-4073 and H-4318, respectively (*pb0.05).

FIG. 8B: Viability of A2780 and HSMC cells exposed to 10 pM H-4073, HO-3867, 3-CPH (N-hydroxy-3-carbamoyl proxyl), or 3-CP (3-carbamoyl proxyl) for 24 h. The results did not show any significant effect of 3-CPH or 3-CP on the cell viability, suggesting that the N-hydroxypyrroline or its nitroxide form is not cytotoxic to either type of cell under the conditions used.

FIGS. 9A-9D: Metabolic conversion and superoxide scavenging of DAPs in cells.

FIG. 9A: Reversible, one-electron oxidation of the —NOH moiety to nitroxide (—NO′), which is paramagnetic and can be detected by EPR spectroscopy.

FIG. 9B: EPR spectra of 10 pM H-4073 in PBS, 10 pM HO-3867 in PBS, and 10 pM HO-3867 after 6-h incubation with A2780 cells at 37° C. The three-line pattern is characteristic of the nitroxide (—NO) metabolite in solution.

FIG. 9C: Amounts of nitroxide upon incubation of 100 pM HO-3867 with various cancer and noncancerous cells for 6 h at 37° C. Control represents the measure of nitroxide in the culture medium (without cells) incubated for 6 h at 37° C. Data represent means±SD (N=5). The data show the presence of a substantial portion of HO-3867 in the nitroxide form in cellular incubations and further that the nitroxide levels in the noncancerous cells are significantly higher (*pb0.05) compared to each of the cancer cells.

FIG. 9D: Superoxide radical-scavenging ability of DAPs. Superoxide radicals were generated using an aerobic solution of xanthine and xanthine oxidase and detected as DEPMPO-OOH adduct by EPR spectroscopy. The DAP compounds (100 NM) were used to compete with 1 mM DEPMPO for the superoxide ions. Superoxide dismutase (SOD; 4.2 pM) was used as a positive control. Data represent means±SD (N=5), *pb0.05 versus control. The results show that the N-hydroxypyrroline-modified DAPs, HO-3867 and HO-4200, are capable of scavenging superoxide radicals

FIG. 10: Intracellular ROS generation by DAPs. A2870 and HSMC cells were incubated with 10 μM DAP (H-4073, HO-2867, H-4318, HO-4200) for 12 h. ROS formation was assessed using the fluorescence dye CM-H2DCF-DA (10 μM). Representative fluorescence images and quantitation (means±SE; N=5) of ROS production in A2780 and HSMC cells are shown. Control refers to untreated cells. *pb0.05 vs the respective A2870 cells. The results demonstrate that the N-hydroxypyrroline-conjugated DAPs, HO-3867 and HO-4200, show significantly lower amounts of ROS in HSMC compared to A2780 cells.

FIG. 11: Inhibition of STAT3 signaling by HO-3867. Cells were treated with HO-3867 for 24 h and subjected to Western blot analysis. Representative immunoblot images of total and phosphorylated (Tyr705 and Ser727) STAT3 and apoptotic markers of cleaved caspase-3 and PARP are shown for A2780, A549, HepG2, MCF-7, HCT-116, and HSMC cells. Note the decreased levels of both pSTAT3 Tyr705 and pSTAT3 Ser727 and corresponding increased levels of cleaved caspase-3 and cleaved PARP in the treated cells compared to untreated cells.

FIG. 12A-12C: HO-3867 inhibits cancer cell migration and invasion. Cell-migration (wound healing) assay was performed by Transwell cell-invasion assay using A2780 and SKOV3 cancer cells at 0 and 24 h, and in the presence of HO-3867 (10 μM) at 24 h.

FIG. 12A: A representative image of six experiments is shown for each group. Gap size and cell invasion were quantified in the regions flanked by dotted lines. The residual gap between the migrating cells from the opposing edges is expressed as a percentage of the initial, scraped area. Data represent mean±SE (N=6); *p<0.05 versus Control (24 h). The migration results show that HO-3867 significantly inhibited the reduction in gap size caused by cell migration.

FIG. 12B: Inhibition of A2780 and SKOV3 cell invasion by HO-3867 (10 μM) at 24 h using a Boyden chamber migration assay. Representative images selected from six experiments are shown for each group. Quantification of cell invasion expressed as a percent of Control. Data represent mean±SE (N=6); *p<0.05 versus Control (24 h). The invasion results show that HO-3867 significantly inhibited cell invasion.

FIG. 12C: Quantitation of the effect of HO-3867 (10 μM; 24-h incubation) on gap-size and cell-invasion of VEGF-induced cell migration and invasion in A2780 and SKOV3 cells. Data represent mean±SE (N=6); *p<0.05 versus VEGF group. The results show that HO-3867 significantly inhibited the effect of VEGF on cell migration and invasion.

FIGS. 13A-13C: FAS and FAK are involved in cancer cell migration and invasion.

FIG. 13A: Basal levels of FAS and FAK expression in human ovarian cancer cell lines.

FIG. 13B: Effect of silencing of FAS and FAK in A2780 cells by transfection of FAS siRNA and FAK siRNA, respectively, on cell migration expressed as a percent of Control. Data represent mean±SE (N=6); *p<0.05 versus Control.

FIG. 13C: Effect of silencing of FAS and FAK in A2780 cells by transfection of FAS siRNA and FAK siRNA, respectively, on cell invasion expressed as a percent of Control. Data represent mean±SE (N=6); *p<0.05 versus Control. The results show that silencing of FAS and FAK significantly inhibited cell migration and invasion.

FIGS. 14A-14C: HO-3867 inhibits FAS and FAK expression in ovarian cancer cells.

FIG. 14A: Representative Western blots showing a time-dependent inhibition of FAS and FAK levels in A2780 and SKOV3 cells treated with HO-3867 (10 μM).

FIG. 14B: Expression levels of FAS and FAK mRNA following HO-3867 exposure (10 μm for 24 h).

FIG. 14C: FAS activity measured in A2780 and SKOV3 cells using NADPH by spectrophotometry. *p<0.05 versus respective control (0 h). The results show that HO-3867 significantly inhibited FAS and FAK expressions in cancer cells.

FIG. 15: HO-3867 downregulates FAS and FAK levels via proteasome pathways. To catch any ubiquitinated proteins in the cell lysates, agarose beads coated with domains having affinity to ubiquitin were incubated in the lysates at 4° C. for 2 hours. After washing the beads, the ubiquitinated proteins were subjected to immunoblot for FAS and FAK and blotted by the ubiquitin antibody. A predominant ubiquitination of FAS and FAK is seen in the HO-3867-treated A2780 and SKOV3 cells under proteasomal inhibition using MG 132 (50 μM). The results show that HO-3867 clearly downregulated the FAS stability protein of USP2a in a time-dependent manner.

FIG. 16: HO-3867 downregulates FAS/FAK regulatory genes. A2780 and SKOV3 cells were treated with HO-3867 (10 μM; 24 h) followed by blotting of FAS/FAK-regulating proteins HER1, SREBP1, ERK1/2, MMP-2 and VEGF. The results show that HO-3867 at 24-h incubation clearly down-regulated the FAS/FAK-regulating proteins in both ovarian cancer cell lines.

FIGS. 17A-17C: HO-3867 suppresses FAS/FAK and VEGF levels in tumor tissues.

FIG. 17A: Tissue lysates containing 50 mg protein of A2780 xenograft tumors from mice treated with 50 or 100 ppm HO-3867, containing 50-μg protein each, were subjected to immunoblot analyses. The decreased expression of FAS, FAK, and VEGF levels are noted in the HO-3867-treated tumor lysates, in a dose-dependent manner.

FIG. 17B: Quantitative results of FAS, FAK and VEGF bands by densitometric analysis. Data represent mean±SE (N=3). *p<0.05 versus untreated (0 ppm) group.

FIG. 17C: Immunohistochemistry showing decreased expression of FAS and VEGF levels in the tumor tissues. The results show that HO3867-treatment to mice suppressed FAS, FAK, and VEGF levels in the tumor.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the subject invention provides methods and compositions altering the amount of a target genome in a target cell. In further describing the subject invention, the subject methods are described first in greater detail, followed by a review of various representative applications in which the subject invention finds use as well as kits that find use in practicing the subject invention

Introduction

There is provided herein compositions comprising a redox based curcumin derivative. In particular, the composition comprising a redox based curcumin derivative, diarylidenylpiperiden-4-one (DAP) having a hydroxylamine moiety attached thereto, and generally having the structure:

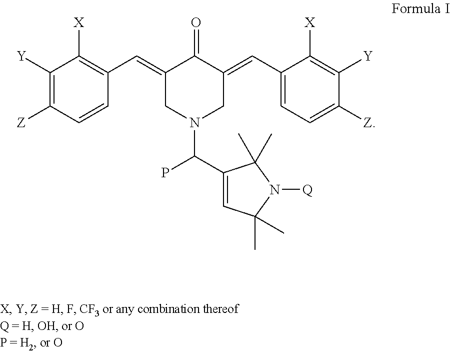

In certain embodiments, the composition has the general structure:

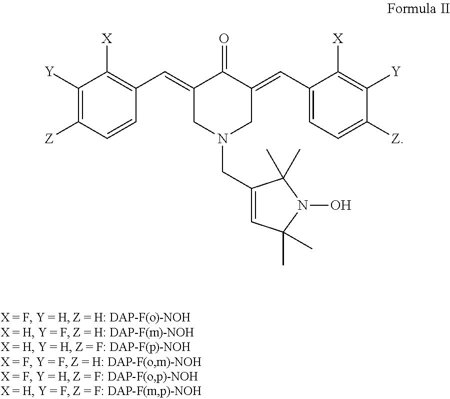

For example, one useful composition is DAP-F(p)-NOH (1-[(1-oxyl-2,2,5,5-tetramethyl-2,5-dihydro-1H-pyrrol-3-yl)methyl]-(3E,5E)-3,5-bis(4-fluorobenzylidene)piperidin-4-one)[HO-3867] having the structure:

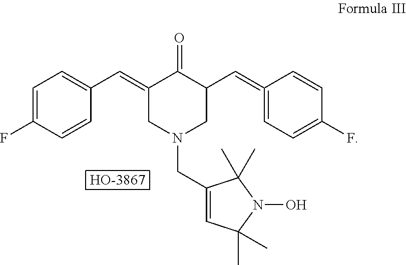

The compositions of Formulae I-III are useful for providing protection to normal cells from associated oxidative damage, while simultaneously providing a desired anti-cancer efficacy.

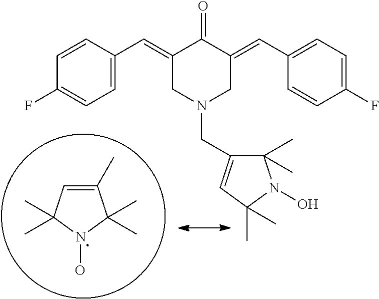

The compositions have in vivo cleavable hydroxylamine formed on an available pyridine group. The compositions are capable of undergoing redox cycling to its correspondent nitroxide form in vivo. Also, the compositions are capable of inducing the generation of reactive oxygen species (ROS) in cancer cells, yet not significantly inducing the generation of ROS in normal cells. As such, the compositions are useful to provide protection to normal cells from associated oxidative damage, while simultaneously providing a desired anti-cancer efficacy.

Administration of the compositions causes a reduction in the cell viability in cancer cells, while having little effect on the viability of normal cells. The compositions thus protect normal cells from associated oxidative damage.

The compositions induce apoptosis in a cell by regulating the down-regulation of suppressors of apoptosis. The compositions regulate the inhibition of the JAK-STAT pathways, and also regulate one or more proteins involved in G₂/M cell cycle arrest.

The compositions attenuate cancer cell migration and invasion through inhibition of FAS and FAK expression, as further explained herein. Also, the compositions not only inhibit cancer cell migration and invasion, but also block VEGF-induced angiogenesis For example, the composition of Formula III, HO-3867, is capable of suppressing the migration and invasion of ovarian cancer cells by inhibiting the expression/activity of motility-promoting proteins including FAS, FAK, and VEGF.

Also provided herein is a method for treatment of cancers and disorders associated with cancers. As further described herein, these compositions have a lower toxicity than a diarylidenylpiperiden-4-one (DAP), while having substantially the equivalent anti-tumor efficacy as DAP-F(p) against various human cancers.

These compositions are thus useful in treating acute or chronic free-radical associated diseases. Also, these compositions are useful in treating and/or lessening the severity of a cancer as these compositions are useful as anti-cancer agents that destroy the cancer cells while leaving surrounding healthy tissues/cells unharmed.

The composition include a covalent coupling of a hydroxylamine moiety to a DAP-F(p) composition. That is, the compositions can be considered a bifunctional composition having a diarylidenyl piperidone (DAP) moiety conjugated to a nitroxide precursor (NOH) moiety, where the DAP moiety has cytotoxic activity, and the NOH moiety functions as a tissue-specific modulator of cytotoxicity.

The compositions described herein have an in vivo cleavable hydroxylamine formed on an available pyridine group. That is, the compositions described herein are capable of undergoing redox cycling to its correspondent nitroxide form in vivo. The compositions have substantially lower toxicity than a diarylidenylpiperiden-4-one (DAP) composition, and further have a substantially equivalent anti-tumor efficacy compared with DAP against cancers. The compositions retain the anti-cancer activity of DAP-F(p) while affording protection to normal cells from associated oxidative damage.

The compositions are useful as anti-cancer agents that are capable of destroying cancer cells while leaving surrounding healthy tissues/cells unharmed

While the inventors do not intend to be bound by theory, the following is a description of the basis for the invention.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

The value of the present invention can thus be seen by reference to the Examples herein.

### EXAMPLES

The present invention is based, at least in part, on the inventors' discovery and determination of: (a) the anticancer efficacy of HO-3867 toward cancerous and a noncancerous (control) cell lines, (b) the action of HO-3867, and (c) whether HO-3867 would significantly inhibit tumor growth in an in vivo model of ovarian cancer. The studies were conducted using human ovarian cancer cell lines and a murine xenograft model of ovarian cancer. The results showed a preferential toxicity of HO-3867 toward ovarian cancer cells and suppression of tumor growth through inhibition of the JAK/STAT3 pathway both in vitro and in vivo.

### Example 1

### Materials and Methods

Chemicals

Curcumin, superoxide dismutase, 6-carboxy-2′,7′-dichlorodihydrofluorescein diacetate, diacetoxy-methyl ester, MIT, and antibodies against actin were obtained from Sigma. 5-Diethoxyphosphoryl-5-methyl-1-pyrroline-N-oxide was from Radical Vision. Cell-culture medium (RPMI 1640), fetal bovine serum, antibiotics, sodium pyruvate, trypsin, and PBS were purchased from Life Technologies. Polyvinylidene fluoride membrane and molecular weight markers were obtained from Bio-Rad. Antibodies against poly-adenosine diphosphate ribose polymerase (PARP), cleaved caspase-3, caspase-7, caspase-8, STAT3, phospho-STAT3 (Tyr705), JAK1, BclxL, and phospho-JAK1 (Tyr1022/1023) were purchased from Cell Signaling Technology. Antibodies specific for cyclin A, cyclin D1, cyclin-dependent kinase (Cdk)2, p53, p21, p27, Fas/CD95, FasL, Bcl-2, and ubiquitin were purchased from Santa Cruz Biotechnology. Enhanced chemiluminescence reagents were obtained from Amersham Pharmacia Biotech (GE Healthcare). All other reagents, of analytical grade or higher, were purchased from Sigma-Aldrich unless otherwise noted.

Synthesis of H-4073 and HO-3867

Melting points were determined with a Boetius micromelting point apparatus and are uncorrected. Elemental analyses (C, H, N, S) were done on a Fisons EA 1110 CHNS elemental analyzer. Mass spectra were recorded on Thermoquest Automass Multi and VG TRIO-2 instruments the EI mode. 1H NMR spectra were recorded with a Varian UNITYINOVA 400 WB spectrometer. Chemical shifts are referenced to Me4Si. Measurements were run at 298K probe temperature in a CDCl₃ solution. Flash column chromatography was done on a Merck Kieselgel 60 (0.040-0.063 mm). Qualitative thin-layer chromatography was carried out on commercially prepared plates (20×20×0.02 cm) coated with Merck Kieselgel GF254. All chemicals were purchased from Aldrich. Compound HO-350 was prepared as described in Hankovzky H O, Hideg K, Lex L. Nitroxyls VII. Synthesis and reactions of highly reactive 1-oxyl-2,2,5,5-tetramethyl-2,5-dihydropyrrole-3-ylmethyl sulfonates. Synthesis 1980:914-6.

### (3E,5E)-3,5-Bis(4-fluorobenzylidene)piperidin-4-one (H-4073)

A solution of 4-fluorobenzaldehyde (2.48 g, 20.0 mmol) and 4-piperidone hydrate hydrochloride (1.53 g, 10.0 mmol) was allowed to stay in glacial acetic acid (saturated with HCl gas previously) for 2 days. The precipitated yellow solid was filtered, washed with Et₂O (30 mL), and the yellow hydrochloride salt 2.50 g (72%; melting point, 212-214° C.) was air dried and used in the next step without further purification. For analytic characterization, 300 mg of the salt were dissolved in water (10 mL) and basified by addition of 250 mg K₂CO₃ and extracted with CHCl₃ (3×10 mL). The combined extracts were dried (MgSO₄), filtered, and evaporated to obtain a yellow solid compound [R_{f}: 0.43 (CHCl₃/Et₂O, 2:1). MS (EI, 70 eV): m/z (%): 311 (M+, 73) 282 (43), 148 (36), 133 (100). Anal Calcd. for: C₁₉H₁₅F₂NO: C, 73.30; H, 4.86; N, 4.50. Found: C, 73.19; H, 4.83; N, 4.32. 1H NMR (399.9 MHz, DMSO-d₆): δ 4.38 (s, 4H), 7.26 (t, 4H), 7.48 (m, 4H), 7.81 (s, 2H)].

### 1-[(1-Oxyl-2,2,5,5-tetramethyl-2,5-dihydro-1H-pyrrol-3-yl)methyl]-(3E,5E)-3,5-Bis(4-fluorobenzylidene)piperidin4-one (HO-3867)

A mixture of H-4073 HCl salt (1.73 g, 5.0 mmol) and K2CO3 (1.38 g, 10.0 mmol) in acetonitrile (20 mL) was stirred at room temperature for 30 minutes. Then, allylic bromide and HO-350 (1.28 g, 5.5 mmol) were added dissolved in acetonitrile (5 mL) and the mixture was stirred and refluxed till the consumption of the starting materials (−3 h). After cooling, the inorganic salts were filtered off on sintered glass filter, washed with CHCl₃ (10 mL), the filtrate was evaporated, and the residue was partitioned between CHCl₃ (20 mL) and water (10 mL). The organic phase was separated; the aqueous phase was washed with CHCl₃ (20 mL); and the combined organic phase was dried (MgSO₄), filtered, and evaporated. The residue was purified by flash column chromatography (Hexane/EtOAc) to obtain the deep yellow solid title compound [1.36 g (59%), R_{f}: 0.57 (Hexane/EtOAc, 2:1), mp 142-144° C. MS (EI, 70 eV): m/z (%): 463 (M+, 12) 433 (20), 324 (40), 310 (43), 133 (100). Electron spin resonance: a_{N}=14.9 G. Anal Calcd. for: C₂₈H₂₉F₂N₂O₂: C, 72.55; H, 6.31; N, 6.04. Found: C, 72.54; H, 6.23; N, 6.04].

To achieve the N-hydroxy compound HCl salt, HO-3867 (1.0 g) was dissolved in ethanol (20 mL, saturated with HCl gas previously) and refluxed for 30 minutes. Then, the solvent was evaporated off and the procedure was repeated till the disappearance of the electron paramagnetic resonance triplet line to give the HCl salt. Stock solutions of the compounds were freshly prepared in DMSO.

Cell Lines and Cultures

The A2780 human epithelial ovarian cancer cell line was used. Ovarian cancer cell lines used (SKOV3, OVCAR3, A2780R, and OV4), as were normal human ovarian surface epithelial (hOSE; ScienCell Ovarian Cell System) cells, were grown in RPMI 1640 and DMEM supplemented with 10% fetal bovine serum, 2% sodium pyruvate, 1% penicillin, and 1% streptomycin. Cells were grown in a 75-mm flask to 70% confluence at 37° C. in an atmosphere of 5% CO2 and 95% air. Cells were routinely trypsinized (0.05% trypsin/EDTA) and counted using an automated counter (NucleoCounter, New Brunswick Scientific).

Cell Viability by MTT Assay

Cell viability was determined by a colorimetric assay using MTT. In the mitochondria of living cells, yellow MTT undergoes a reductive conversion to formazan, producing a purple color. Cells, grown to −80% confluence in 75-mm flasks, were trypsinized, counted, seeded in 96-well plates with an average population of 7,000 cells per well, incubated overnight, and then treated with curcumin, H-4073, or HO-3867 for 24 hours. All experiments were done using eight replicates and, repeated at least thrice.

Cell Proliferation by Clonogenic Assay

Cell survival was assessed by clonogenic assay. Cells at −80% confluence were trypsinized, rinsed, seeded onto 60-mm dishes (5×10⁴ cells per dish), grown for 24 hours at 37° C., and treated afterward with H-4073 or HO-3867 for 24 hours. Nontreated cells served as controls. After treatment, the cells were washed twice with PBS, trypsinized, counted, and plated in 60-mm dishes in triplicate and incubated for an additional 7 days. The colonies were then stained with crystal violet (in ethyl alcohol) and counted using an automated colony counter (ColCount, Oxford Optronix). Each experiment was repeated at least five times.

Cell-Cycle Analysis

Cells were treated with HO-3867 (10 μmol/L) for 24 hours, trypsinized, washed in PBS, and fixed in an ice-cold 75% ethanol/PBS solution. The DNA was labeled with propidium iodide. Cells were sorted by flow cytometry and cell cycle profiles were determined using ModFit LT software (Becton Dickinson).

Immunoblot Analysis

Cells in RPMI 1640 were treated with DMSO (control) or HO-3867 (10 μmol/L) for 24 hours. Equal volumes of DMSO (0.1% v/v) were present in each treatment. Following treatment, the cell lysates were prepared in non-denaturing lysis buffer containing 10 mmol/L Tris-HCl (pH 7.4), 150 mmol/L NaCl, 1% Triton X-100, 1 mmol/L EDTA, 1 mmol/L EGTA, 0.3 mmol/L phenylmethyl-sulfonyl fluoride, 0.2 mmol/L sodium orthovanadate, 0.5% NP40, 1 μg/mL aprotinin, and 1 μg/mL leupetin. The lysates were centrifuged at 10,000×g for 20 minutes at 4° C. and the supernatant was separated. The protein concentration in the lysates was determined using a Pierce detergent-compatible protein assay kit. For Western blotting, 25 to 50 μg of protein lysate per sample were denatured in 2×SDS-PAGE sample buffer and subjected to SDS-PAGE on a 10% tris-glycine gel. The separated proteins were transferred to a polyvinylidene fluoride membrane and were blocked with 5% nonfat milk powder (w/v) in TBST (10 mmol/L Tris, 10 mmol/L NaCl, 0.1% Tween 20) for 1 hour at room temperature or overnight at 4° C. The membranes were then incubated with the primary antibodies. The bound antibodies were detected with horseradish peroxidase-labeled sheep anti-mouse IgG or horseradish peroxidase-labeled donkey anti-rabbit IgG using an enhanced chemiluminescence detection system (ECL Advanced kit). Protein expressions were determined using the Image Gauge v. 3.45 software.

Ovarian Cancer Tumor Xenografts in Mice

Cultured A2780 cancer cells (2×10⁶ cells in 60 pL of PBS) were s.c. injected into the back of 6-week-old BALB/c nude mice from the National Cancer Institute. Five to 7 days later, when the tumors reached 3 to 5 mm in diameter, the mice were divided (n=9/group) in a manner to equalize the mean tumor diameter among the groups. The control group was given a normal diet (no treatment), whereas the experimental groups were treated using the DAP compounds mixed with the animal feed (Harlan Teklad) at three different levels (25, 50, and 100 ppm). The doses were chosen based on an initial dose-response study optimized to produce an observable effect on tumor growth. The size of the tumor was measured twice per week using a digital Vernier caliper. The tumor volume was determined from the orthogonal dimensions (d1, d2, d3) using the formula (d₁×d₂×d₃)×π/6. Thirty five days after the beginning of HO-3867 treatment, the mice were sacrificed and the tumors were resected. The tumor tissues were then subjected to immunoblot analysis.

Data Analysis

The statistical significance of the results was evaluated using ANOVA and a Student's t test. A P value of <0.05 was considered significant.

Results for Example 1

HO-3867 is Cytotoxic to A2780 and Other Ovarian Cancer Cell Lines.

The cytotoxic effects of H-4073 and HO-3867 were evaluated and compared with that of curcumin in A2780 and other established human ovarian cancer cell lines.

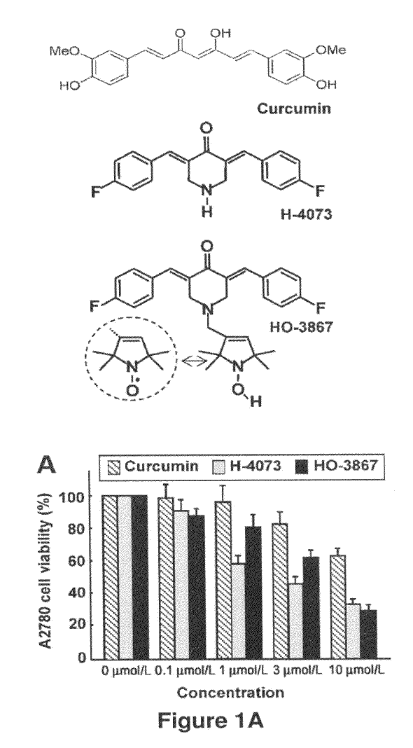

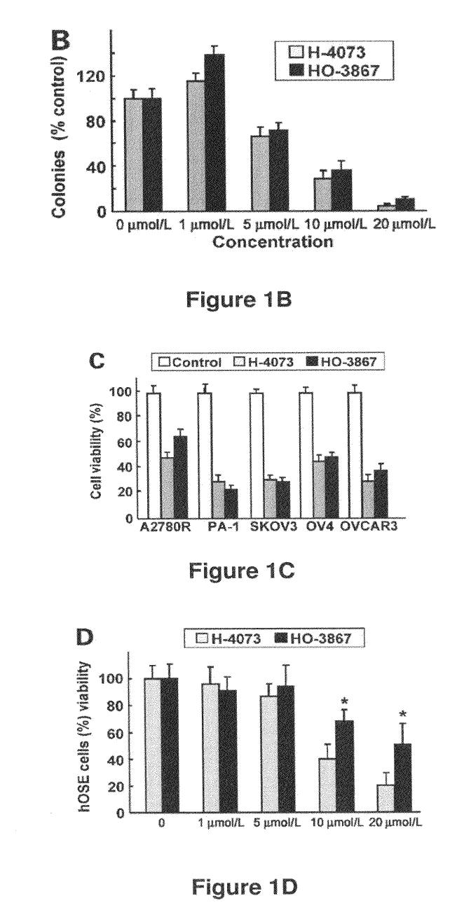

FIG. 1A compares the effect of curcumin, H-4073, and HO-3867 on the viability of A2780 cells. Although all three compounds showed a dose-dependent cytotoxicity, H-4073 and HO-3867 exhibited significantly higher toxicity when compared with curcumin. The results further indicated that the cytotoxic effects of HO-3867 and H-4073 on A2780 cells were comparable, showing that the introduction of the N-hydroxypyrroline moiety in HO-3867 did not compromise the cytotoxic effect of HO-3867 against A2780 cells.

We next did clonogenic assays to study the effectiveness of H-4073 and HO-3867 on the proliferation of A2780 cells. Both compounds showed a dose-dependent reduction in the number of colonies (FIG. 1B), showing that the compounds are equally potent in inhibiting cell proliferation.

We further tested the cytotoxicity of H-4073 and HO-3867 in a number of other well-established human ovarian cancer cell lines including a cisplatin-resistant derivative of A2780 (A2780R), PA-1, SKOV3, OV4, and OVCAR3. The results (FIG. 1C) showed that both H-4073 and HO-3867 were equally and significantly toxic to the tested cell lines.

We then tested the effect of HO-3867 exposure on hOSE cells, which are noncancerous control cell lines derived from human ovarian surface epithelium. As shown in FIG. 1D, no significant cytotoxicity to hOSE cells was discovered for up to 10 μmol/L concentration of HO-3867. However, treatment with 20 μmol/L H-4073 or HO-3867 showed significant cytotoxicity to hOSE cells. Taken together, the cellular viability studies showed that both H-4073 and HO-3867 were comparably and significantly effective in inducing cytotoxicity in A2780 and other ovarian cancer cell lines; however, HO-3867 was significantly less toxic to noncancerous hOSE cells when compared with H-4073.

HO-3867 Induces G2-M Cell Cycle Arrest in A2780 Cells

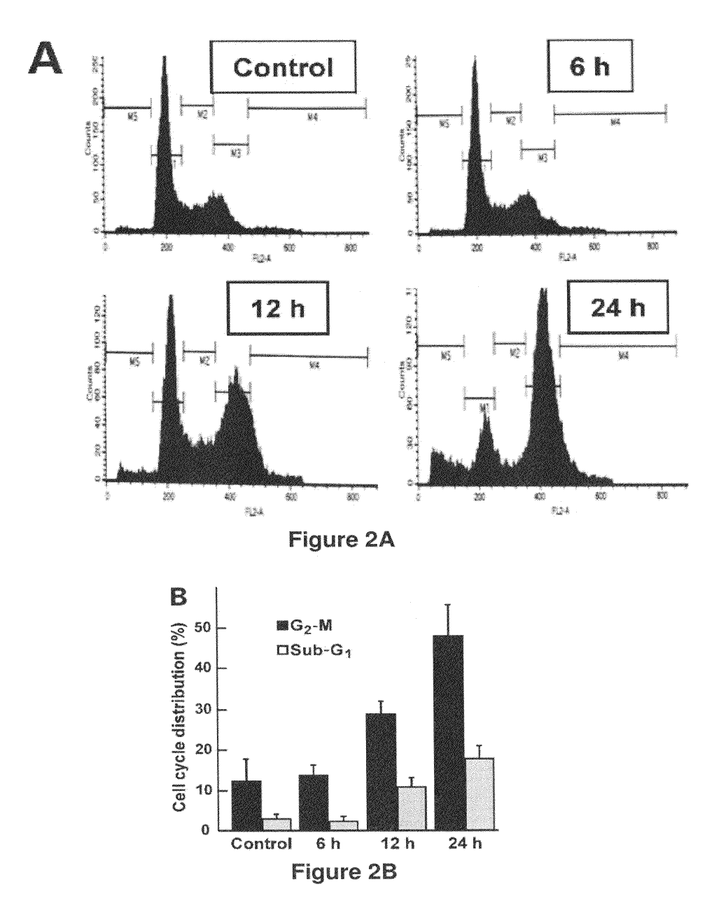

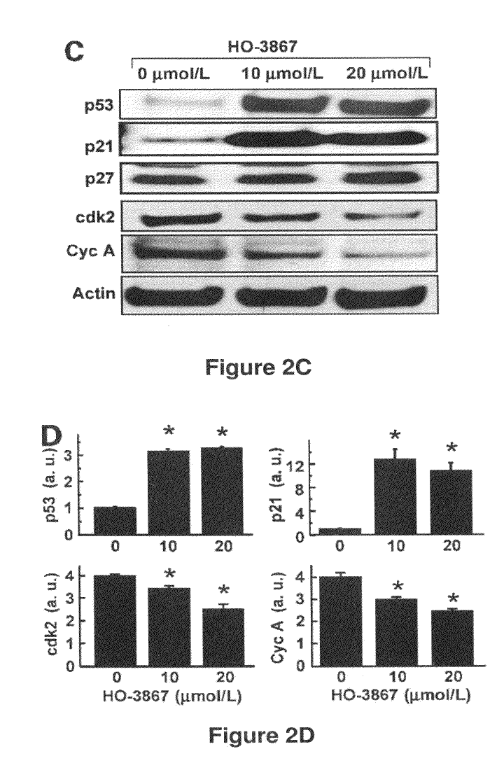

We next examined whether the growth inhibition of A2780 cells by HO-3867 was caused by cell cycle arrest. Cells were treated with HO-3867 for 6, 12, or 24 hours; fixed; and cell cycle populations were determined by flow cytometry. The results showed that the percentages of the cell population in the G2-M and sub-G1 phases were significantly higher in the treatment group when compared with the nontreated control group (FIG. 2A and FIG. 2B).

We then determined the effect of HO-3867 on the cell cycle regulatory molecules p53, p21, p27, cdk2, and cyclin A (FIG. 2C) by Western blotting. The levels of p53 and p21 were significantly upregulated, whereas cdk2 and cyclin-A levels were significantly decreased after treatment (FIG. 2D). These results indicated that HO-3867 caused G2-M cell cycle arrest, at least in part, by modulating cell cycle regulatory proteins.

HO-3867 Induces Apoptosis in A2780 Cells

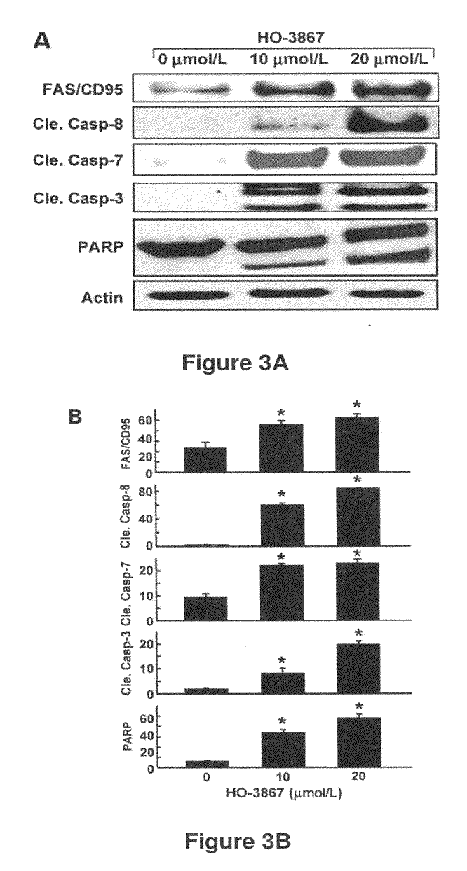

The arrest of cell cycle progression in cancer cells is usually associated with the concomitant activation of proapoptotic pathways. To determine whether HO-3867-induced cell cycle arrest led to apoptosis, the expression of activated caspases were probed by Western blotting. The blots showed the activation of caspase-8, caspase-7, cleaved caspase-3, and PARP in A2780 cells treated with HO-3867 for 24 hours (FIG. 3A). The quantitative results of the immunoblots (FIG. 3B) showed a significant increase in the level of these caspases in cells treated with HO-3867 when compared with nontreated cells.

We further determined the levels of caspase-8-associated death receptors such as Fas/CD95 and Fas-L. The data from the Fas/CD95 expression showed a clear increase in HO-3867-treated cells when compared with nontreated group. However, no significant change was discovered in Fas-L (data not shown).

HO-3867 Inhibits the JAK/STAT3 Pathway

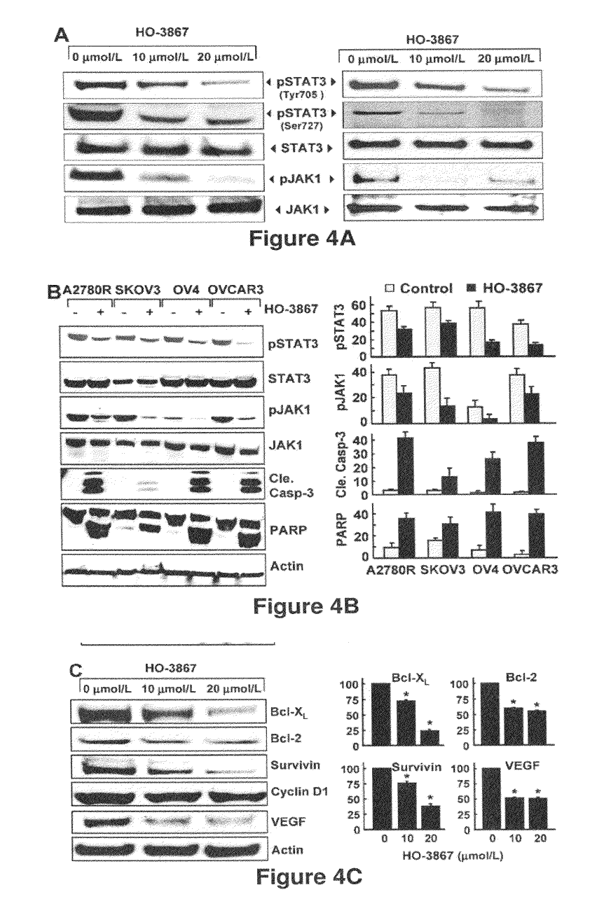

The constitutive activation of STAT3 in ovarian cancer has been shown to regulate the expression of genes implicated in tumor-cell proliferation and survival. To determine whether the HO-3867-induced growth inhibition in A2780 cell was mediated by STAT3, we examined the level of phosphorylated STAT3 (pSTAT3) by Western blotting (FIG. 4A). The pSTAT3 (Tyr705 and Ser727) levels were significantly decreased after treatment with 10 or 20 μmol/L HO-3867 for 24 hours.

Excessive JAK activity in tumor cells is one of the most common mechanisms for constitutive activation of STAT3. To examine whether HO-3867 exposure resulted in a decrease in STAT3 activation through JAK kinase inhibition, we measured the phosphorylated level of JAK1 after 24 hours of exposure to 10 or 20 μmol/L HO-3867 (FIG. 4A). In addition, we cross-checked the expression of these proteins using immunoprecipitation and confirmed the decreased expression of pSTAT3 and pJAK1 with no change in the total expression levels of these proteins. The data showed a substantial decrease in phosphor-JAK1 (Tyr1022/1023) when compared with unexposed controls, suggesting that HO-3867 blocked the JAK/STAT3 pathway. Before proceeding to in vivo experiments, we confirmed the potent apoptosis-inducing effect of HO-3867 in four additional ovarian cancer cell lines, A2780R, SKOV3, OV4, and OVCAR3. Following HO-3867 treatment, all four cell lines clearly showed caspase-3 and PARP cleavage, accompanied by a decrease in the expression levels of pSTAT3 and JAK1 (FIG. 4B). The results suggested that induction of apoptosis and inhibition of JAK/STAT3 signaling could be caused by HO-3867 in human ovarian cancer cell lines.

HO-3867 Downregulates the STAT3 Target Proteins

To investigate the downstream consequences of STAT3 inhibition, Western blotting was done to determine the protein levels of Bcl-xL, Bcl-2, survivin, vascular endothelial growth factor (VEGF), and cyclin D1 following 24-hour exposure to HO-3867 at 10 and 20 μmol/L concentrations. There was a clear reduction in Bcl-xL, Bcl-2, survivin, and VEGF levels, whereas no significant change in cyclin D1 level was discovered (FIG. 4C).

Because these proteins are involved in anti-apoptotic (survivin, Bcl-2, and Bcl-xL) and angiogenic (VEGF) protein expressions, the downregulation of these proteins suggested profound antitumor potential of HO-3867 in ovarian cancer cells. The results clearly showed the involvement of the STAT3 pathway in the growth inhibition of the A2780 ovarian cancer cells by HO-3867.

HO-3867 Inhibits the Growth of Xenograft Tumor in Mice

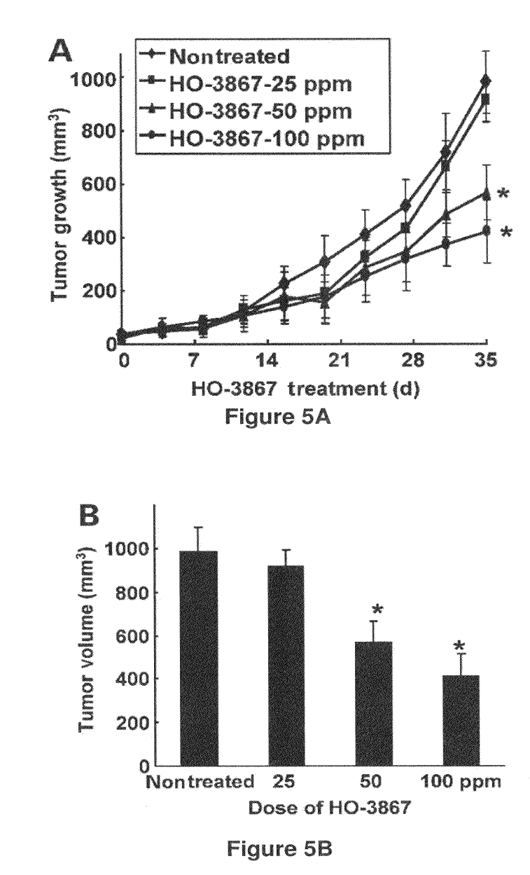

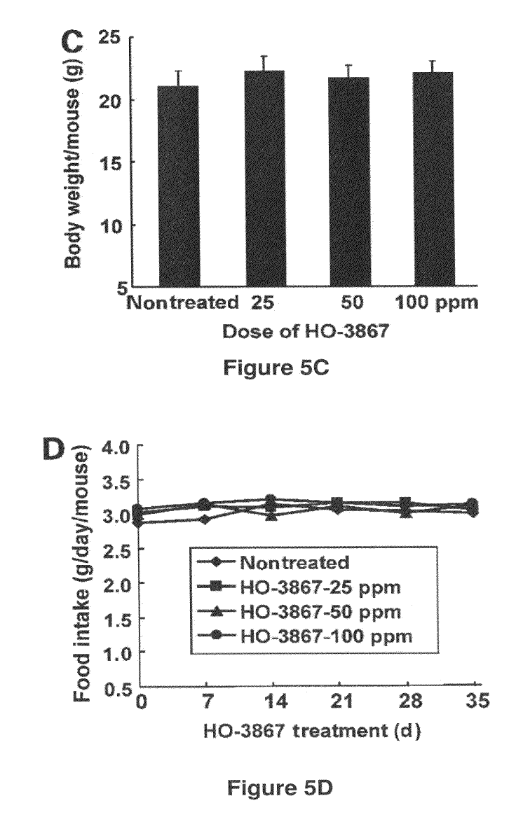

Based on the in vitro results, which showed significant cytotoxicity of HO-3867 to human ovarian cancer cell lines, we next evaluated the efficacy of HO-3867 in a human ovarian tumor xenograft grown in the back of mice. The mice were treated with HO-3867 and the tumor size as measured twice weekly for 5 weeks. A significant reduction in the tumor volume was discovered in a dose-dependent manner; particularly, the doses of 50 and 100 ppm were more effective when compared with vehicle-treated controls (FIG. 5). We also measured the body weight and diet consumption of tumor-bearing animals. HO-3867-treated animals did not show any gross signs of toxicity and/or possible adverse side effects as measured by two profiles: body weight (FIG. 5C) and diet consumption (FIG. 5D). These results show that the in vivo antitumor efficacy of HO-3867 against ovarian tumor without any apparent signs of toxicity.

HO-3867 Inhibits pSTAT3 and Downregulates the STAT3-Targeting Proteins In Vivo

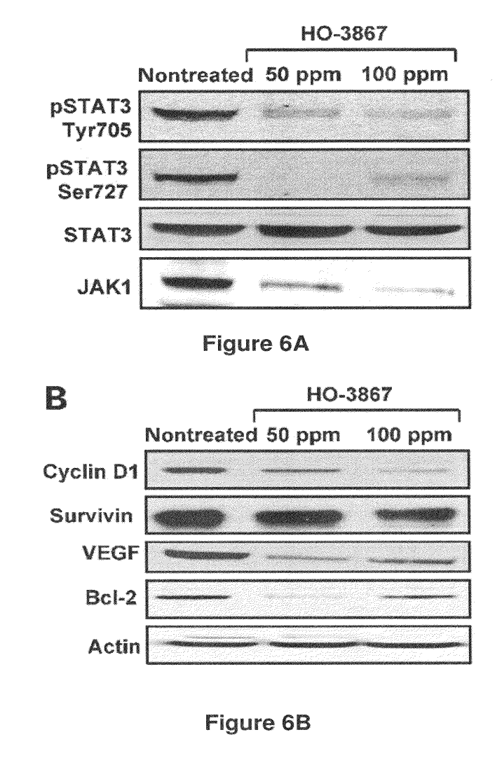

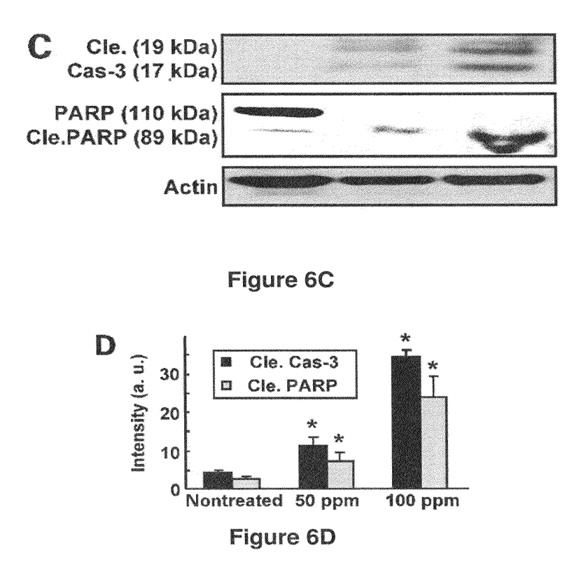

We further analyzed the excised tumor tissue to determine whether HO-3867 inhibited the STAT3/JAK protein expression levels, as discovered in the in vitro studies. In concert with the decreased expressions in both pSTAT3 Tyr705 and Ser727 levels, without affecting total STAT3, we discovered a clear downregulation of total JAK levels in tumor tissues. However, we did not observe any significant change in pJAK levels in tumor tissues (FIG. 6A). As for the target gene products of STAT3, we discovered a clear decrease of cyclin D1, VEGF, Bcl-2, and Survivin levels in the HO-3867-treated tumors (FIG. 6B). Interestingly, we noticed a significant induction of cleaved caspase-3 and PARP, which is a known marker of apoptosis and a downstream target of activated caspase-3 (FIG. 6C and FIG. 6D), showing that HO-3867 induced apoptosis in vivo.

Discussion of Example 1

These results establish that the di-fluorodiarylidenyl piperidone, HO-3867, exhibits potent anticancer efficacy toward human ovarian cancer cells and xenograft tumors. HO-3867, which also incorporates an antioxidant function, exhibits substantially lower toxicity toward noncancerous cells. The mechanistic studies revealed that HO-3867 targets multiple pathways, including altering the proteins involved in G2-M cell cycle arrest, increased expression of Fas/CD95, downregulation of antiapoptotic signals, and inhibition of the JAK/STAT3 pathway in both in vitro and in vivo. Cell cycle control plays a critical role in the regulation of tumor cell proliferation. Many cytotoxic agents arrest cell cycle at the G1, S, or G2-M phase. In the present study, HO-3867 induced G2-M cell cycle arrest in A2780 cells as evidenced by a significant increase in the p53, p21, and p27 protein levels.

We also discovered a significant reduction in Cdk2 and cyclin A levels. The G2-M-phase progression is regulated by a number of Cdk/cyclins as well as Cdk inhibitors such as p21 and p27. These results show that the HO-3867-induced G2-M cell cycle arrest is mediated by the induction of p53 and p21 and downregulation of cyclin A and Cdk2.

Many curcumin derivatives induce apoptosis in cancer cells, but the mechanisms by which they do so differ. The death receptor-associated mechanism has been recently receiving much attention for the anticancer activity of curcumin derivatives. We discovered that the death receptor gene Fas/CD95 was activated in A2780 cells by HO-3867. We further discovered that the expression level of TNF-R1, the receptor of tumor necrosis factor-α, was unchanged in the HO-3867-treated A2780 cells (data not shown). It has been reported that curcumin promoted tumor necrosis factor-α-induced apoptosis in a variety of cancer cells, but without a significant increase in the TNF-R1 expression level. Curcumin and curcumin analogues have also been shown to upregulate death receptor 5 and FasL expression, thereby inducing apoptosis in human cancer cells. Thus, these results show a critical involvement of upregulated death receptor super-family-mediated signals in the stimulation of A2780 apoptosis following HO-3867 exposure.

STAT3 has been shown to suppress the transcription of Fas/CD95. This suggests the HO-3867-mediated downregulation of STAT3 expression, in both in vitro and in vivo, as a putative mechanism for increased Fas/CD95 expression. This is can now be seen from the substantial decrease in the level of Tyr705-pSTAT3, a major active form of activated STAT3. It is noteworthy that the expression level of Ser727-pSTAT3 was also clearly decreased in vivo. Because the Ser727 phosphorylation is also known to regulate the transcriptional activity of STAT3, this attenuated phosphorylation is suggested to participate in the downregulation of the transcriptional activity of STAT3 in the xenograft tumor treated with HO-3867.

We also discovered that HO-3867 caused a substantial inhibition of phospho-JAK1 (Tyr-1022/1023), suggesting that HO-3867 can inhibit the constitutive activation of STAT3, which may be caused, at least in part, by the inhibition of pJAK1. However, HO-3867 may also inhibit STAT3 activation through JAK2, Src, Erb2, and epidermal growth factor receptor, which are implicated in STAT3 activation as well.

Downstream proteins of STAT3 have been shown to regulate apoptosis and regulation in cancer cells. For example, Bcl-xL, Bcl-2, and survivin have been shown to suppress apoptosis, whereas c-myc and cyclin D1 have been shown to mediate proliferation. Because of the fact that STAT3-downregulating genes are all critically involved in the development of cancer aggressiveness, targeting STAT3 is considered a potential anticancer strategy.

Furthermore, inhibition of STAT3 expression in vivo has provided deep insight into a new approach for the treatment of human tumors. In addition, inhibition of STAT3 activation is valid in inducing significant apoptosis in both the mice model of melanoma xenografts and that of squamous cell carcinoma xenografts.

The induction of apoptosis in tumor is another approach to limit their uncontrolled proliferation of tumor growth. In this process, activation of caspases is the central event. Once activated, the executioner caspases downstream of the cascade act on the key molecules inside the cells to orchestrate cell death.

The present Example 1 shows that HO-3867 clearly induces apoptotic death both in vitro and in vivo, at least in part, due to the activation of caspase-3 and cleavages of PARP. Cleavage of PARP by activated caspases is considered as a marker for apoptotic death. STAT3 is also known to protect cells from apoptosis through the upregulation of Bcl-xL, Bcl-2, and survivin. The expression levels in all of these molecules downstream of STAT3 activation were clearly reduced in ovarian cancer cells by exposure to HO-3867, not only in vitro but also in vivo—even in mice given a low concentration (50 ppm) of HO-3867. This implies that induction of apoptosis may be an additional contributing factor in the HO-3867-mediated inhibition of ovarian tumor growth. However, further studies are essential to elucidate the mechanism of apoptosis induction by HO-3867.

Also, the Example 1, shows that the anticancer efficacy of a novel curcuminoid compound, HO-3867, inhibited ovarian tumor growth by inhibition of the JAK1/STAT3 signaling pathway. HO-3867 is useful as a therapeutic agent for treating ovarian cancer.

### Example 2

### Materials and Methods

Chemicals

Superoxide dismutase (SOD), 6-carboxy-2¹,7¹-dichlorodihydrofluorescein diacetate, diacetoxymethyl ester (H2DCF-DA), 3-(4,5-dimethylthiazol-2-yl)-2,5-diphenyltetrazolium bromide (MTT), and antibodies against actin were obtained from Sigma (St. Louis, Mo., USA). 5-Diethoxyphosphoryl-5-methyl-1-pyrroline-N-oxide (DEPMPO) was from Radical Vision (Jerome-Marseille, France). Cell culture medium (RPMI 1640), fetal bovine serum (FBS), antibiotics, sodium pyruvate, trypsin, and phosphate-buffered saline (PBS) were purchased from Gibco (Grand Island, N.Y., USA). Polyvinylidene difluoride (PVDF) membrane and molecular weight markers were obtained from Bio-Rad (Hercules, Calif., USA). Antibodies against poly(adenosine diphosphate ribose) polymerase (PARP), cleaved caspase-3, STAT3, and phospho-STAT3 (Tyr705) were purchased from Cell Signaling Technology (Beverly, Mass., USA). Enhanced chemiluminescence reagents were obtained from Amersham Pharmacia Biotech (Piscataway, N.J., USA). The DAPs used, namely, H-4073 ((3E,5E)-3,5-bis(4-fluorobenzylidene)piperidin-4-one), HO-3867 (1-[(1-oxyl-2,2,5,5-tetramethyl-2,5-dihydro-1H-pyrrol-3-yl)methyl]-(3E,5E)-3,5-bis(4-fluorobenzylidene)piperidin-4-one), H-4318 ((3E,5E)-3,5-bis(4-trifluoromethylbenzylidene)piperidin-4-one), and HO-4200 (1-[(1-oxyl-2,2,5,5-tetramethyl-2,5-dihydro-1H-pyrrol-3-yl)-methyl]-(3E,5E)-3,5-bis(4-trifluoromethylbenzylidene)piperidin-4-one), were synthesized. Stock solutions of the compounds were freshly prepared in dimethyl sulfoxide (DMSO). All other reagents, of analytical grade or higher, were purchased from Sigma-Aldrich, unless otherwise noted.

Cell Lines and Cultures

The A2780 human epithelial ovarian cancer cell line and human aortic smooth muscle cell line (HSMC) were used. Other cancer cell lines used were A2780R (cisplatin-resistant human ovarian cell line), A549 (human lung cancer cell line), HepG2 (human liver cancer cell line), HCT-116 (human colon cancer cell line), PC3 (human prostate cancer cell line), MCF-7 (human breast cancer cell line), and SCC4 (human squamous cell carcinoma cell line). Also used were the following noncancerous (healthy) cell lines: human ovarian surface epithelial cells (hOSE; ScienCell Ovarian Cell System) and human aortic endothelial cells (HAEC).

The cells were grown in the following media: cancer cells in RPMI 1640 or DMEM, HSMC and HAEC in SmBM, hOSE cells in OEPiCM). The medium was supplemented with 10% FBS, 2% sodium pyruvate, 1% penicillin, and 1% streptomycin. Cells were grown in a 75-mm flask to 70% confluence at 37° C. in an atmosphere of 5% CO2 and 95% air. Cells were routinely trypsinized (0.05% trypsin/EDTA) and counted using an automated counter (NucleoCounter, New Brunswick Scientific, Edison, N.J., USA).

Cell Counting

Untreated and DAP compound-treated cells were counted using a NucleoCounter (New Brunswick Scientific).

Electron Paramagnetic Resonance Spectroscopy

Electron paramagnetic resonance (EPR) spectroscopy was used to quantitatively determine the relative superoxide-scavenging ability of the DAPs in comparison with DEPMPO, a known superoxide scavenger. Superoxide radicals were generated using an aerated solution of PBS (pH 7.4) containing xanthine (0.2 mM), xanthine oxidase (0.02 U/ml), and diethylenetriamine pentaacetic acid (0.1 mM). The superoxide radicals (O₂⁻) generated by the xanhine-xanthine oxidase reaction were reacted with DEPMPO (1 mM) to form a paramagnetic adduct (DEPMPO-OOH), which was detected using X-band (9.8 GHz) EPR spectroscopy. In separate experimental groups, H-4073 (100 pM), HO-3867 (100 pM), H-4318 (100 pM), HO-4318 (100 pM), or SOD (500 U/ml) was added to the reaction mixture. If the DAPs could scavenge superoxide radicals, then there would be a competition between these compounds and DEPMPO for reaction with superoxide, and hence the EPR intensity of the DEPMPO-OOH adduct could serve as a measure of the superoxide-scavenging ability of the test compounds. The generation of the DEPMPO-OOH adduct was measured at 10 min after initiation of the reaction.

Cell Viability by MTT Assay

Cell viability was determined by a colorimetric assay using MTT. In the mitochondria of living cells, yellow MTT undergoes a reductive conversion to formazan, giving a purple color. Cells, grown to −80% confluence in 75-mm flasks, were trypsinized, counted, seeded in 96-well plates with an average population of 7000 cells/well, incubated overnight, and then treated with the DAPs (H-4073, HO-3867, H4318, or HO-4200; 10 pM) for 24 h. All experiments were done using eight replicates and repeated at least three times. Cell viability was expressed as the percentage of MIT viability of untreated cells.

Measurement of Intracellular ROS

The ROS levels in cells treated with DAPs were determined using H2DCF-DA, a membrane-permeative fluorogenic probe. The acetate and acetoxymethyl ester groups of this probe are enzymatically cleaved inside living cells. The probe can then be oxidized by intracellular oxidants (ROS) to give a product, DCF, which emits a strong, green fluorescence (λₑₓ=504 nm; λₑₘ=529 nm). The fluorescence intensity is proportional to the level of cellular oxidants. Cells, grown to 80% confluence on 6-mm glass coverslips, were treated with 10 pM H-4073, HO-3867, H-4318, or HO-4200 for 12 h, followed by incubation with H2DCF-DA. The cells were further incubated in the dark for 20 min and washed with protein-free medium, and then fluorescence images were immediately captured with a Nikon Eclipse TE2000-U camera system using excitation/emission at 495/520 nm. The captured images were then analyzed using MetaMorph image analysis software.

Western Blot Assay

Cells were grown in RPMI 1640 medium and treated with DMSO (control) or HO-3867 (10 pM). Equal volumes of DMSO (0.1% v/v) were present in both groups. After treatment, cell lysates were prepared in nondenaturing lysis buffer (10 mM Tris-HCl (pH 7.4), 150 mM NaCl, 1% Triton X-100, 1 mM EDTA, 1 mM EGTA, 0.3 mM phenylmethylsulfonyl fluoride, 0.2-mM sodium orthovanadate, 0.5% NP-40, 1 lag/ml aprotinin, and 1 lag/ml leupeptin). Cell lysates were centrifuged at 10,000 RPM for 20 min at 4° C., and the supernatant was separated. The protein concentration in the lysates was determined using a Pierce detergent-compatible protein assay kit. For Western blotting, 25 to 50 lag of protein lysate per sample was denatured in 2× sample buffer and subjected to SDS-PAGE on a 10 or 12% Tris-glycine gel. The separated proteins were transferred to a PVDF membrane and then the membrane was blocked with 5% nonfat milk powder (w/v) in 10 mM Tris, 100 mM NaCl, 0.1% Tween 20 for 1 h at room temperature or overnight at 4° C. The membranes were incubated with the primary antibodies described above. The bound antibodies were detected with horseradish peroxidase (HRP)-labeled sheep anti-mouse IgG or HRP-labeled donkey anti-rabbit IgG (Amersham) using an enhanced chemiluminescence detection system (ECL Advanced Kit). Protein expression was determined using Image Gauge version 3.45.

Statistical Analysis

Data are expressed as means±SEM. Comparisons among groups were performed using the Student t test. The significance level was set at pb0.05.

Results for Example 2

Cytotoxicity of DAPs to Cancer Cells

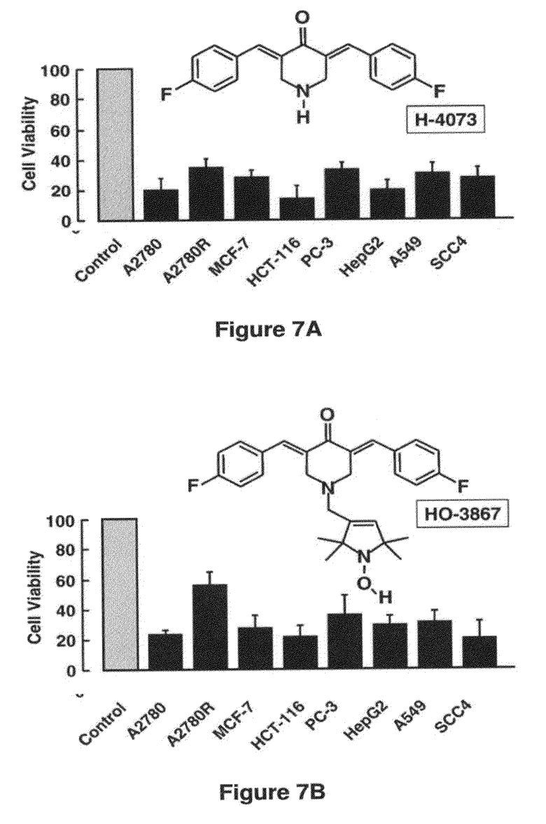

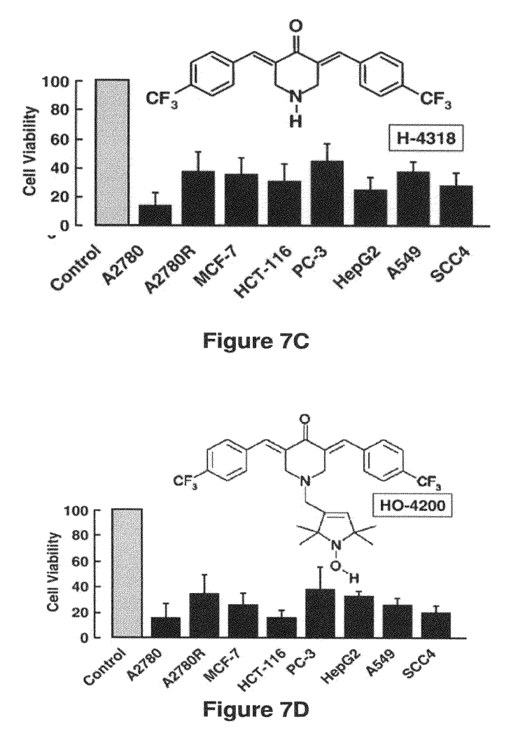

The cytotoxicity of DAPs (H-4073, HO-3867, H-4318, HO-4200) to established human cancer cell lines, namely A27820, A2780R, MCF-7, HCT-116, PC-3, HepG2, A549, and SCC4, was evaluated by exposing the cells to 10 pM concentration of the compounds for 24 h. All four compounds induced a substantial loss of cell viability in all the human cancer cell lines tested (FIG. 7).

In particular, H-4073 and H-4318 exhibited higher toxicity compared to HO-3867 or HO-4200. The results further indicated that the DAPs were more cytotoxic to ovarian (A2780) and colon (HCT-116) cancer cells compared to other cancer cells tested.

Cytotoxicity of DAPs to Noncancerous Cells

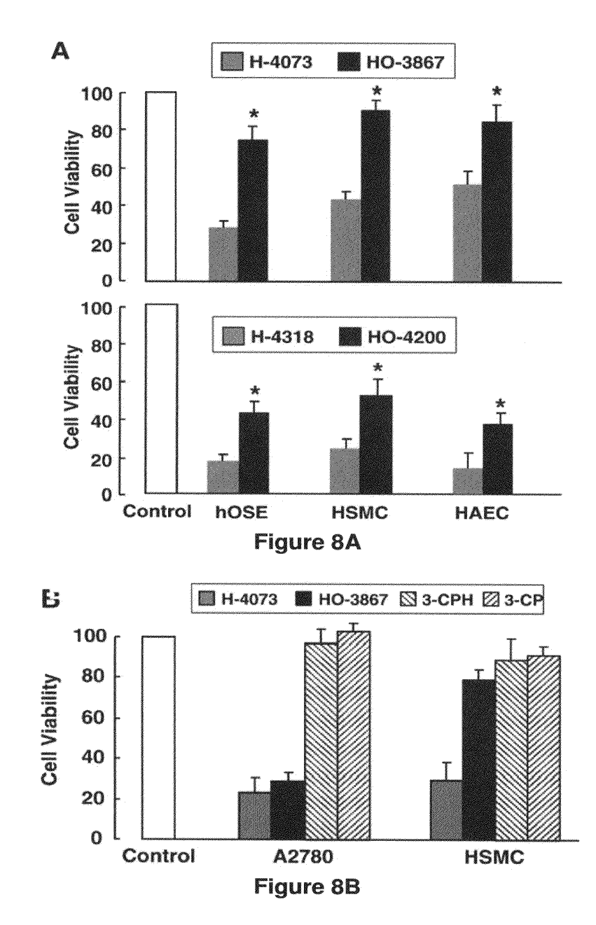

We next compared the effect of DAPs (10 NM; 24-h incubation) on the viability of noncancerous (healthy) human cell lines, namely hOSE cells, HSMC, and HAEC. All four compounds, in general, induced a substantial loss of cell viability in the cells tested, although to different extents (FIG. 8A).

The N-hydroxypyrroline-appended DAPs, HO-3867 and HO-4200, were significantly less toxic to the healthy cells compared to H-4073 and H-4318, respectively. In particular, the results of HO-3867 showed a strikingly differential effect on cancer versus noncancerous cells. While not wishing to be bound by theory, the inventors herein now believe that this differential effect stems from the N-hydroxypyrroline function.

To determine the role of the N-hydroxypyrroline function in the cytotoxicity, we additionally evaluated the effects of 3-CPH (a stand-alone analog of N-hydroxypyrroline) and 3-CP (a nitroxide version 3-CPH) on A27180 and HSMC cells. The results did not show any significant effect of 3-CPH or 3-CP on the cell viability (FIG. 8B), showing that the N-hydroxypyrroline or its nitroxide form is not cytotoxic to either type of cell under the conditions used. Overall the viability results implicate the diarylidenyl piperidone group in inducing cytotoxicity and the N-hydroxypyrroline group in protecting noncancerous cells.

Metabolic Conversion of DAPs in Cells

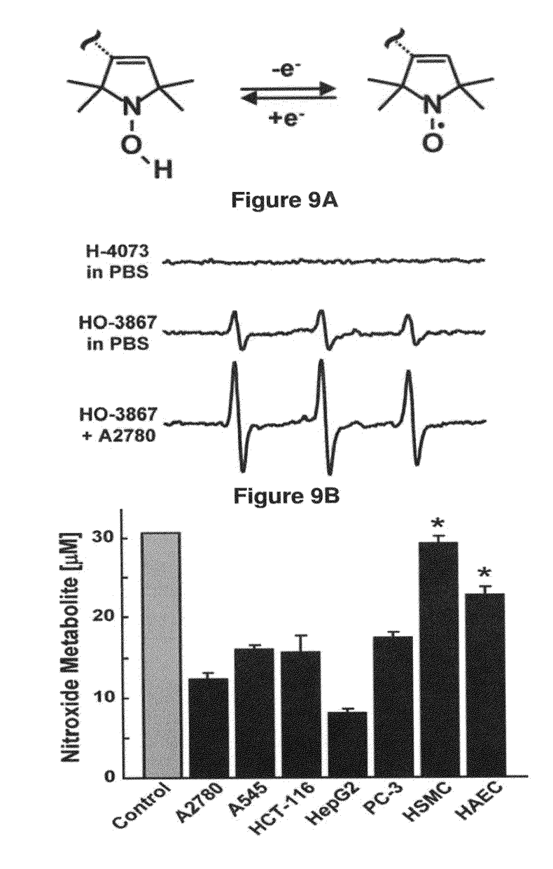

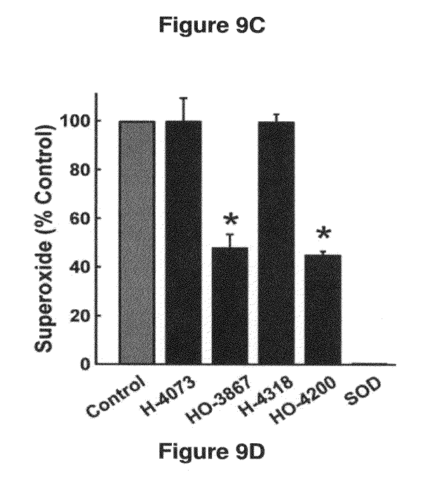

The N-hydroxypyrroline (—NOH) moiety is capable of undergoing a reversible one-electron oxidation to its nitroxide form (—NO; FIG. 9A), which is paramagnetic and detectable by EPR spectroscopy. Hence, we next determined whether HO-3867 and HO-4200 are converted to their corresponding nitroxide form in cells. The EPR spectrum measured from a 100 pM solution of HO-3867 incubated with A2780 cells showed a characteristic triplet feature (FIG. 9B) attributable to nitroxide, as verified by using an authentic nitroxide form of HO-3867 (data not shown). A fivefold increase in the EPR signal intensity of the nitroxide metabolite was discovered in A2870 cells incubated with HO-3867 compared to PBS. Similar results were obtained with HO-4200 (data not shown). Under these conditions, H-4073 and HO-4318 did not show any EPR signal, showing that the N-hydroxypyrroline moiety is the source of the discovered EPR signal. FIG. 9C shows the nitroxide metabolite levels upon incubation of cells with 100 μM HO-3867 at 37° C. for 6 h. The results show the presence of a significant level of the nitroxide form in cells tested and that the metabolite level was significantly higher (25-30%) in noncancerous cells compared to cancer cells (7-16%).

Superoxide Radical-Scavenging Activity of DAPs

Both N-hydroxypyrroline and nitroxide have antioxidant properties including SOD- and catalase-mimetic activity. Therefore, we determined the superoxide radical-scavenging ability of DAPs using a competitive reaction in the presence of DEPMPO. Superoxide radicals were generated using an aerobic solution of xanthine and xanthine oxidase and detected as DEPMPO-OOH adduct by EPR spectroscopy. The DAP compounds (100 μM) were used to compete with 1 mM DEPMPO for the superoxide ions. SOD (4.2 μM) was used as a positive control. HO-3867 and HO-4200 treatment showed a substantial diminution (−50%) of the DEPMPO-OOH concentration, indicative of the scavenging of superoxide radicals (FIG. 9D).

In contrast, H-4073 and H-4318 did not show any significant effect on the superoxide adduct level. The EPR studies clearly demonstrated that the N-hydroxypyrroline-modified DAPs are capable of scavenging superoxide radicals.

ROS Levels in Cells Treated with DAPs

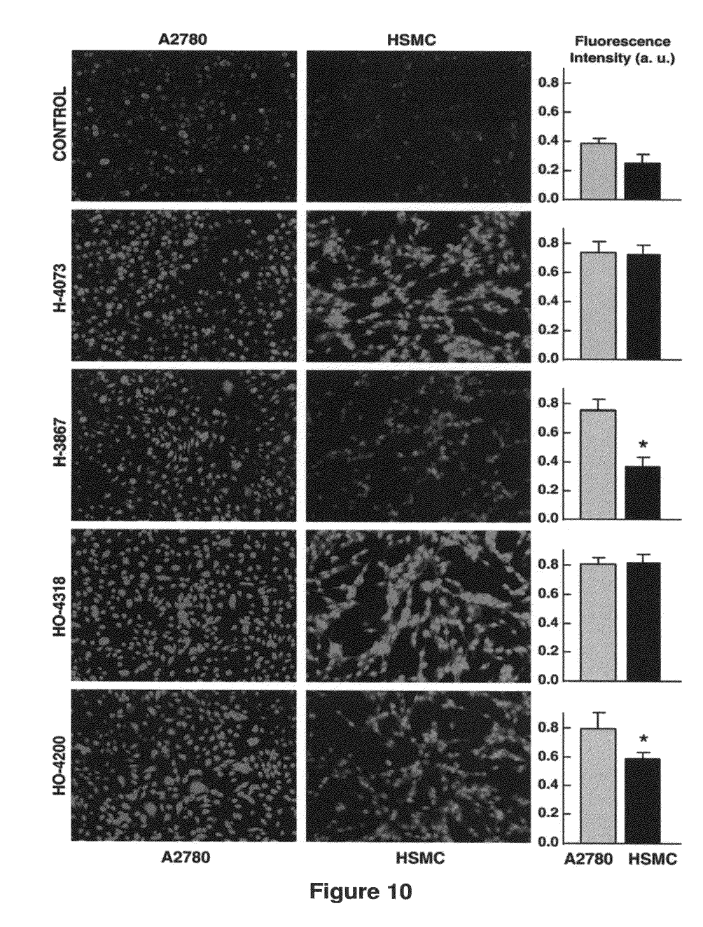

The cytotoxicity of diarylidenyl ketones, such as curcumin and its analogs, is associated with ROS generation in cells. Hence, we determined whether the DAPs could have a similar effect upon cancer cells. A2780 cells were incubated with 5 or 10 μM DAPs for 12 h and intracellular ROS generation was measured by DCF fluorescence. The fluorescence intensity discovered in A2780 cells was significantly higher in the cells treated with DAPs compared to untreated controls (FIG. 10).

In contrast, the DCF fluorescence intensity in HSMC treated with HO-3867 was not significantly different from that of the untreated cells. H-4073, HO-4318, and HO-4200 induced significant ROS generation in both A2780 and HSMC cells. These results show that H-4073 and HO-3867 are comparable in inducing ROS generation in A2780 cells. However, in HSMC, HO-3867 generated significantly less ROS compared to H-4073 and HO-4318. Taken together, the results imply that HO-3867 is capable of inducing oxidative stress in cancer cells while sparing healthy cells.

Effect of HO-3867 on Markers of Cell Proliferation and Apoptosis

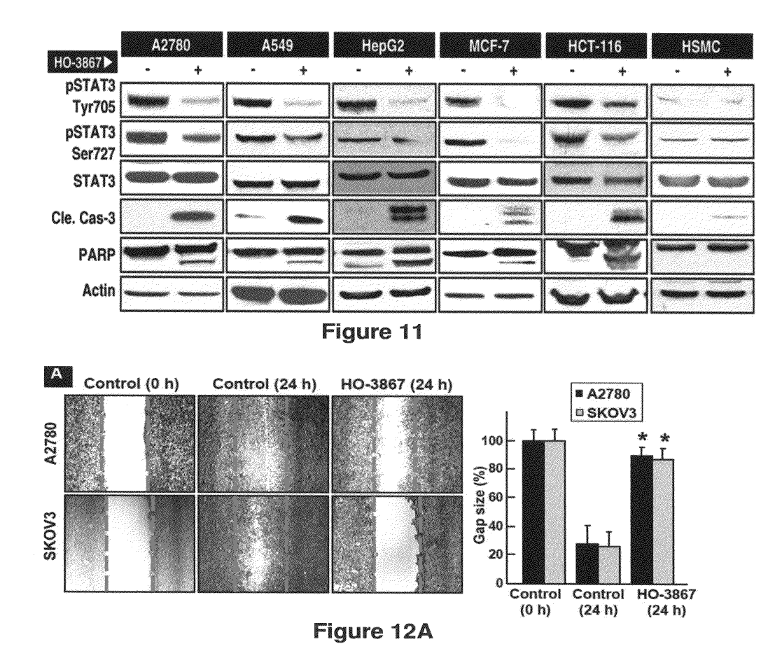

Constitutive activation of STAT3 regulates the expression of genes implicated in the proliferation, survival, and inhibition of apoptosis in cancer cells. Inhibition of STAT3 phosphorylation seems to be a key in the anticancer activity of diarylidenyl ketones. To determine whether the DAP-induced loss of cell viability in this study was mediated by STAT3, we determined the level of phosphorylated STAT3 (Tyr705 and Ser727) by Western blot analysis. The data (FIG. 11) show that both the Tyr705 and the Ser727 pSTAT3 levels were significantly attenuated in cancer cells treated with 10 μM HO-3867 for 24 h. We also discovered significant elevation of activated caspase-3 and PARP in the treated cells, suggesting the induction of apoptosis by inhibition of STAT3 signaling in the HO-3867-exposed cancer cells. It should be noted that HO-3867 did not induce any significant change in the activation of STAT3 or apoptotic markers in HSMC.

Discussion of Example 2

The development of smart anticancer agents that selectively destroy cancer cells while sparing the surrounding healthy tissues/cells is the main goal of cancer therapy. The results of this Example 2 show that all four DAPs induce potential cytotoxicity in cancer cells.

The N-hydroxypyrroline-conjugated DAPs, HO-3867 and HO-4200, although equally toxic to cancer cells, are significantly less toxic to noncancerous (healthy) cells. The differential cytotoxicity is mediated through inhibition of STAT3 activation in cancer cells while providing antioxidant protection to healthy cells. These results show that the antioxidant-conjugated DAPs are useful as safe and effective anticancer agents for cancer therapy.

In biological tissues, nitroxides are reduced to their corresponding hydroxylamine forms (FIG. 9A). These two forms of nitroxide coexist in tissues. Nitroxides can be reduced to the corresponding hydroxylamines by reductants such as ascorbate, glutathione, and semiquinone radicals and also by intercepting reducing equivalents from the electron-transport chain. The hydroxylamines, on the other hand, can be oxidized to nitroxides in the presence of hydrogen peroxide and other oxidants such as transition metal complexes. The redox transformation of the nitroxide/hydroxylamine is tissue specific. Nitroxides confer greater protection to normal tissues than to tumor tissues. This tissue specificity may be due to the fact that the nitroxide remains in the oxidized form in healthy, well-oxygenated tissues but is reduced to its hydroxylamine form in hypoxic tissues, such as those in tumors.

In this Example 2, it was discovered, by using EPR measurements, that HO-3867 and HO-4200 were apparently able to undergo redox cycling to their corresponding nitroxide forms in vitro. We examined the role of the nitroxide moiety by comparing HO-3867 and HO-4200 with their parent forms, H-4073 and H-4318, respectively. We discovered that treatment with HO-3867 and HO-4200 induced a significant loss of cell viability in all cancer cells tested. The decrease in the viability of cancer cells upon HO-3867 treatment was comparable to that of H-4073. It should be noted that the cytotoxic effects of H-4073 and HO-3867 on A2780 cells were significantly higher compared to curcumin under similar conditions. Further, HO-3867 had very little effect on the viability of hOSE cells, whereas H-4073, at the same dose, significantly reduced the viability of hOSE cells.

Low-molecular-weight nitroxides are used in clinical trials of cancer treatment. The nitroxides have no anticancer efficacy, but they are used as protectors of normal tissue against chemotherapy- or radiation-induced cytotoxicity. These results in Example 2, using low-molecular-weight nitroxides, namely 3-CP and 3-CPH, did not show any cytotoxicity toward A2780 or HSMC cells, showing that the anticancer efficacy of HO-3867 or HO-4200 is not due to the pyrroline function.

On the other hand, the decreased cytotoxicity of these compounds in the healthy cells may be due to the protective (antioxidant) nature of the nitroxide metabolite, which is present in significantly higher levels in noncancerous cells than in cancer cells (FIG. 9C). The SOD-mimetic activity of these compounds is comparable to that of the manganese complexes of diacetylcurcumin that have been shown to have similar radical-scavenging properties. Both HO-3867 and HO-4200 induced a significantly higher level of ROS in A2780 cells compared to HSMC (FIG. 10). Although the mechanism of induction of ROS in these cells by DAPs is not known, the relatively lower levels of ROS in the noncancerous cells seems to be due to the elevated levels of nitroxide metabolite. Overall, the differential cytotoxicity of HO-3867 discovered in the cancer and noncancerous cells seems to be due the nitroxide metabolite levels.

The Western blot analyses show that HO-3867 induces apoptosis in cancer cells. The results in Example 2 show that HO-3867, whose structure has close similarity to the diketones, inhibited pSTAT3 Tyr705 and Ser727 in all five cancer cell lines tested (FIG. 11). These results further show that HO-3867 activates cleaved caspase-3 and induction apoptotic markers of PARP in the cancer cell lines, showing that HO-3867 induces apoptosis in human cancer cells by targeting STAT3 proteins.

This Example 2 shows that the DAPs induce potential cytotoxicity in cancer cells while sparing noncancerous cells. The differential cytotoxicity is mediated through the inhibition of STAT3 activation in cancer cells while providing antioxidant protection to the healthy cells. These results show that the antioxidant-conjugated DAPs are useful as safe and effective anticancer agents for cancer therapy.

### Example 3

Example 3 shows the effect of HO-3867 on the migratory ability of ovarian cancer cells and the mechanistic pathways including the involvement of FAS, FAK, and associated signaling proteins. This was performed using two established human ovarian cancer cell lines, namely, A2780 and SKOV3 under in vitro as well as in vivo conditions on xenografted tumor in mice.

The results of Example 3 clearly demonstrate that HO-3867 suppressed the migration and invasion of the ovarian cancer cells by inhibiting the expression/activity of FAS and FAK proteins. The results of Example 3 also show that molecular targeting of FAS and FAK by HO-3867 is useful as a strategy for ovarian cancer therapy.

Materials and Methods

Materials

Cell-culture medium (RPMI 1640) and DMEM, fetal-bovine serum (FBS), antibiotics, sodium pyruvate, trypsin, and phosphate-buffered saline (PBS) were purchased from Gibco (Grand Island, N.Y.). Polyvinylidene fluoride (PVDF) membrane and molecular-weight markers were obtained from Bio-Rad (Hercules, Calif.). Antibodies against, pHER1, HER1, FAS, pERK1/2, ERK1/2, actin, and USP2a were purchased from Cell Signaling Technology (Beverly, Mass.). Antibodies specific for SREBP1, FAK, MMP-2, VEGF, USP2a, and ubiquitin were purchased from Santa Cruz Biotechnology (Santa Cruz, Calif.). Enhanced chemiluminescence (ECL) reagents were obtained from Amersham Pharmacia Biotech (GE Healthcare, Piscataway, N.J.). HO-3867 was synthesized in the laboratory. Stock solutions of the compounds were freshly prepared in dimethylsulfoxide (DMSO). All other reagents, of analytical grade or higher, were purchased from Sigma-Aldrich.

Cell Lines and Cultures

A2780 and SKOV3 human epithelial ovarian cancer cell lines were used. The cells were grown in RPMI 1640 and DMEM medium supplemented with 10% FBS, 2% sodium pyruvate, 1% penicillin and 1% streptomycin. Cells were grown in a 75-mm flask to 70% confluence at 37° C. in an atmosphere of 5% CO₂ and 95% air. Cells were routinely trypsinized (0.05% trypsin/EDTA) and counted using an automated counter (NucleoCounter, New Brunswick Scientific, Edison, N.J.).

Cell Migration and Invasion Assays

Cell-migration assay was performed by wound-healing method. Cells were plated at equal density and grown to 90% confluence. Wounds were created using a sterile pipette tip. Cells were then rinsed with medium and replaced with the fresh medium and incubated with HO-3867 (10 μM). Areas of wound were marked and photographed at various time-points with a phase-contrast microscope. Cell-invasive assay was measured by an in vitro Boyden chamber assay. Briefly, 1×10⁵ cells in 0.5 ml of serum-free RPMI 1640 medium were added to the wells of 8-μm-diameter pore membrane Boyden chambers, either coated with (BD Biosciences, Franklin Lake, N.J.) or without (Corning, Corning, N.Y.) Matrigel. Cells were allowed to invade for 24 hours. Cells that had not penetrated the filters were removed by scrubbing with cotton swabs. Chambers were fixed in 100% methanol for 2 min, stained in 0.5% crystal violet for 2 min, rinsed in PBS and examined using a bright-field microscope. Values for invasion were obtained by counting five fields per membrane and represented as the average of three independent experiments performed over multiple days.

Fatty Acid Synthase Activity Assay

The FAS activity was determined spectrophotometrically at 37° C. in particle-free supernatants by measuring the decrease of absorption at 340 nm due to oxidation of NADPH.

Immunoblot Analysis

Cells in RPMI 1640 medium were treated with DMSO (control) or HO-3867 (10 μM) for 24 h. Equal volumes of DMSO (0.1% v/v) were present in each treatment. Following treatment, the cell lysates were prepared in nondenaturing lysis buffer containing 10-mM Tris-HCl (pH 7.4), 150-mM NaCl, 1% Triton X-100, 1-mM EDTA, 1-mM EGTA, 0.3-mM phenylmethylsulfonyl fluoride, 0.2-mM sodium orthovanadate, 0.5% NP40, 1-μg/ml aprotinin, and 1-μg/ml leupetin. The lysates were centrifuged at 10,000×g for 20 min at 4° C., and the supernatant was separated. The protein concentration in the lysates was determined using a Pierce detergent-compatible protein assay kit. For Western blotting, 25 to 50 μg of protein lysate per sample was denatured in 2×SDS-PAGE sample buffer and subjected to SDS-PAGE on a 10% tris-glycine gel. The separated proteins were transferred to a PVDF membrane and blocked with 5% nonfat milk powder (w/v) in TBST (10-mM Tris, 10-mM NaCl, 0.1% Tween 20) for 1 h at room temperature or overnight at 4° C. The membranes were then incubated with the primary antibodies. The bound antibodies were detected with horseradish peroxidase (HRP)-labeled sheep anti-mouse IgG or HRP-labeled donkey anti-rabbit IgG using an enhanced chemiluminescence detection system (ECL Advanced kit). Protein expressions were determined using Image Gauge version 3.45.

Reverse Transcription-PCR

Total RNA isolated from ovarian tumor tissue was prepared with TRIzol (Life Technologies, Grand Island, N.Y.) according to the manufacturer's instructions. RNA quantification was done using spectrophotometry. Reverse transcription (RT)-PCR analysis for the mRNA expressions in FAS, FAK, VEGF and p21 and the internal control GAPDH was carried out using a GeneAmp PCR System Veriti thermo cycler (Applied Biosystems, Foster City, Calif.) under the following conditions: initial denaturation at 94° C. for 2 min, 35 cycles of amplification (denaturation at 94° C. for 30 s, annealing at 50° C. for 30 s, and extension at 72° C. for 30 s), and extension at 72° C. for 5 min. The PCR products were electrophoresed on 1.5% agarose gel and stained with ethidium bromide.

Ovarian Cancer Tumor Xenografts in Mice

A2780 cells (5×106 cells in 60 μl of PBS) were subcutaneously (s.c.) injected into the back of 6-week-old BALB/c nude mice from the National Cancer Institute. On the 5th day when the tumor size reached approximately 2 to 4 mm, the control groups was supplemented a normal diet (no treatment) while the experimental groups were treated using the DAP compounds mixed with the animal feed (Harlan Teklad) at 2 different levels (500 and 100 ppm). The doses were chosen based on an initial dose-response study optimized to produce an observable effect on tumor growth. The tumor tissues were then subjected to immunoblotting and immunohistochemistry.

Immunohistochemistry

Tumor tissues were fixed in formalin and embedded in paraffin. Sections (6-μm thick) were obtained and used for hematoxylin and eosin staining. For immunofluorescence staining, the tissue sections (8-μm thick) were serially rehydrated in 100%, 95%, and 80% ethanol after deparaffinization with xylene. Slides were kept in steam for 30 min and then washed in PBS (pH 7.4) three times for 5 min each. Tissue sections were incubated with 2% goat serum and 5% bovine serum albumin in PBS to reduce nonspecific binding. The sections were then incubated for 4 h with an anti-mouse anti-FAS, or anti-VEGF. The sections were then incubated with secondary antibodies (1:1000 dilutions) conjugated to horseradish peroxidase (HRP)-labeled sheep anti-mouse IgG or HRP-labeled donkey anti-rabbit IgG (Amersham Pharmacia Biotech). The tissue slides were visualized using a Nikon fluorescence microscope.

Data Analysis

The statistical significance of the results was evaluated using Student's t-test. A p value of less than 0.05 was considered significant.

Results for Example 3

Effect of HO-3867 on Ovarian Cancer Cell Migration and Invasion

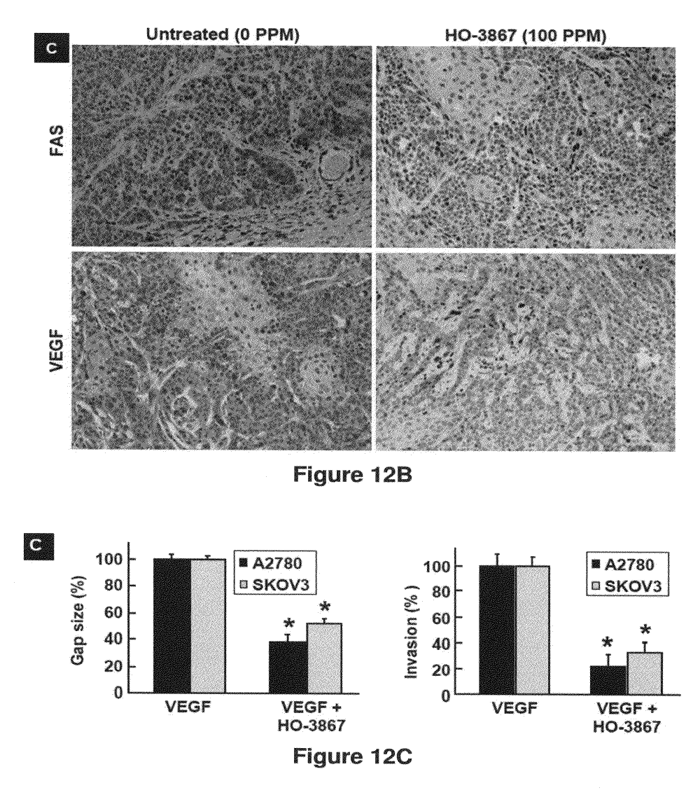

The effect of HO-3867 on the motility of ovarian cancer cells was measured by wound-healing migration and Transwell cell-invasion assays. Incubation of A2780 or SKOV3 cells with HO-3867 (10 μM) for 24 hours showed significant inhibition of cell migration (FIG. 12A) and invasion (FIG. 12B) when compared to untreated cells. Since VEGF-induced angiogenesis is initiated by cell migration and invasion, we next determined whether HO-3867 could inhibit the cell motility-promoting effect of VEGF. We discovered that HO-3867 significantly inhibited the VEGF-induced migration and invasion of both the ovarian cancer cell lines tested (FIG. 12C). The results show that HO-3867 could not only inhibit ovarian cancer cell migration and invasion, but also block VEGF-induced angiogenesis.

Effect of FAS and FAK on Ovarian Cancer Cell Migration and Invasion

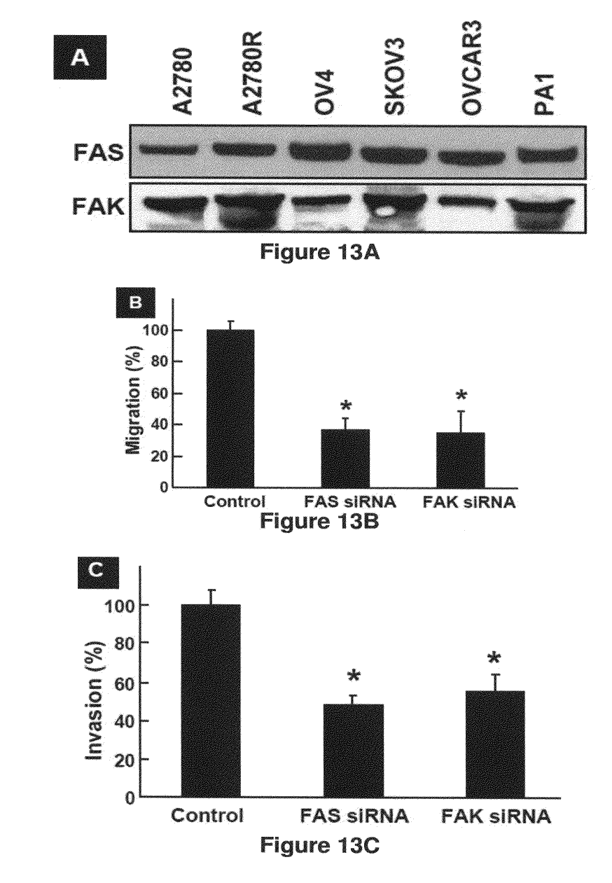

FAS and FAK proteins were significantly expressed in all six human ovarian cancer cell lines tested, including the cisplatin-resistant cancer cell line A2780R (FIG. 13A). We next determined the effect of FAS and FAK inhibition by using their siRNA transfection on the migration and invasion of A2780 cells. Cells transfected with FAS siRNA or FAK siRNA exhibited significant reduction in migration and invasion when compared to control cells. These results show that both FAS and FAK are involved in ovarian cancer cell migration and invasion.

Effect of HO-3867 on FAS and FAK Expression in Ovarian Cancer Cells

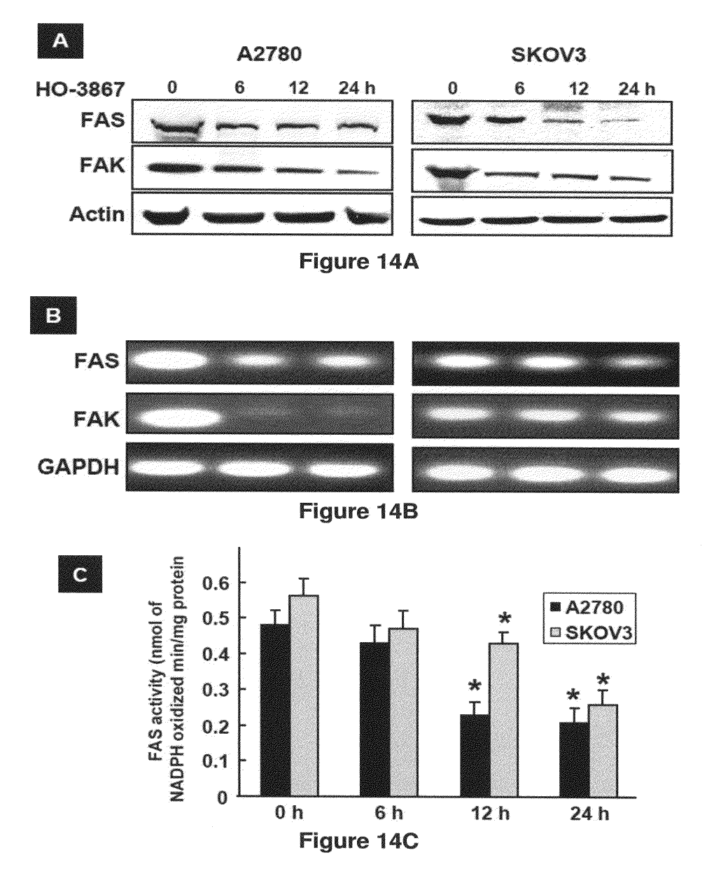

We next determined whether the inhibitory effect of HO-3867 on the ovarian cancer cell migration and invasion was due to its regulation of FAS and FAK expression levels. Incubation of A2780 or SKOV3 cells with HO-3867 (10 μM) resulted in an incubation-time-dependent inhibition of FAS and FAK, both at the protein and expression levels (FIG. 14A, FIG. 14B). We further checked the activity of FAS in cells treated with HO-3867 for 6, 12 and 24 h. The FAS activity was significantly reduced in both the cell lines up on incubation with HO-3867 for 12 or 24 h (FIG. 14C). These results show that HO-3867 inhibited the expression of FAS and FAK in the ovarian cancer cells.

Effect of HO-3867 on FAS and FAK Downregulation by Proteasome Pathways

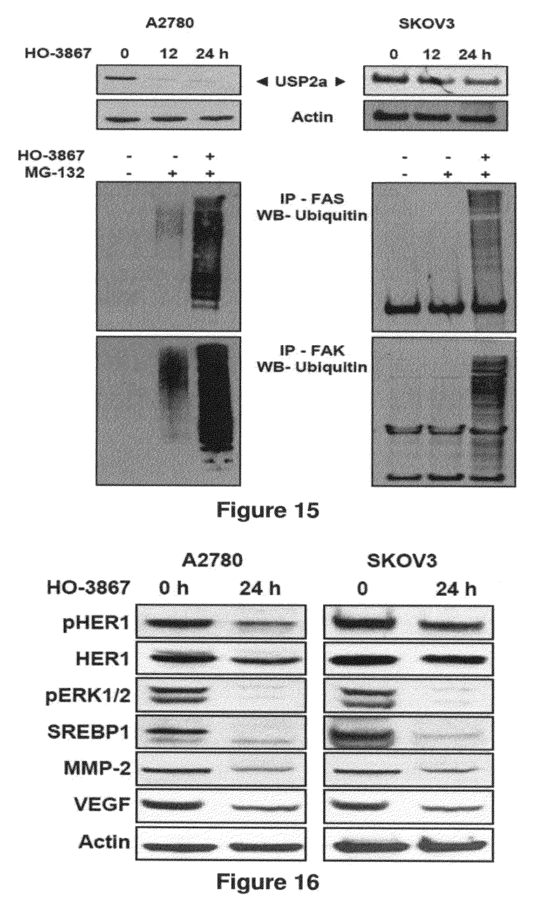

The intracellular amount of rapid-turnover proteins, such as FAS and FAK, is tightly regulated by the ubiquitin-dependent proteolytic pathway. The fast and significant decrease in the FAS and FAK expressions observed in the HO-3867-treated cells prompted us to check any involvement of ubiquitin-dependent degradation mechanism. The isopeptidase USP2a has been shown to regulate the stability of FAS in prostate cancer. We analyzed the USP2a level in HO-3867-treated ovarian cancer cells and discovered that it was clearly downregulated by HO-3867 (FIG. 15). We further observed an enhanced polyubiquitination on FAS and FAK in the HO-3867-treated cancer cells, under the condition of co-incubation with MG-132, a proteasome inhibitor. These results strongly show that HO-3867 inhibited FAS/FAK through the ubiquitination and inhibited FAS stability through USP2a.

Effect of HO-3867 on FAS/FAK Regulatory Genes

We determined the effect of HO-3867 on the FAS and FAK-regulatory cell-migration and invasion genes. HO-3867 inhibited pHER2, pERK1/2, SREBP1, MMP-2, and VEGF in A2780 and SKOV3 cells (FIG. 16). The results show that HO-3867 not only inhibited FAS and FAK expression, but also blocked their regulating genes in the two ovarian cancer cell lines tested.

Effect of HO-3867 on FAS/FAK Levels in Tumor Tissues

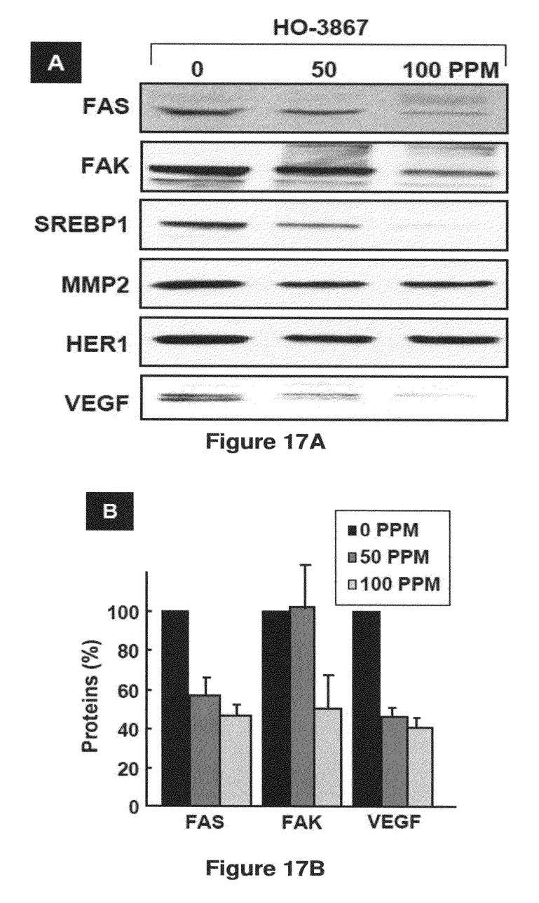

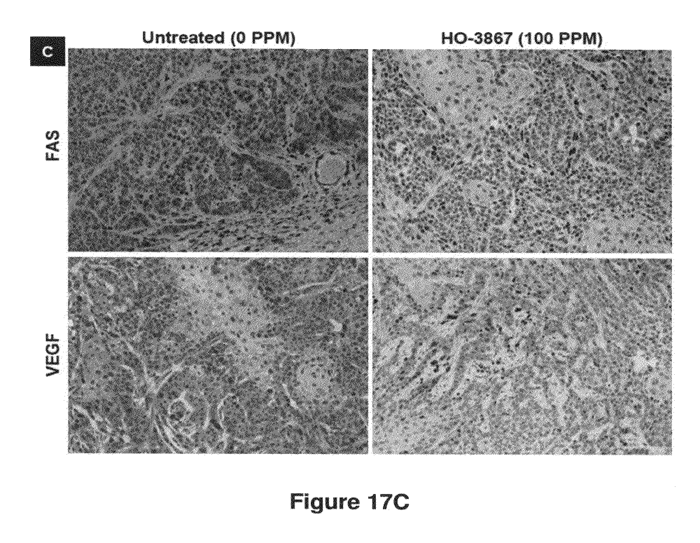

We analyzed the FAS, FAK and their regulatory protein expression levels in an in vivo xenograft mouse model of ovarian cancer. We observed, in concert with the decreased expression of both FAS and FAK, a clear down-regulation in the target gene products of FAS and FAK, namely SREBP1, MMP-2, and VEGF in the xenografted tumor tissues of the HO-3867-treated mice, in a dose-dependent manner (FIG. 17A, 17B). We further determined the FAS and VEGF levels by immunocytochemistry. HO-3867 treatment significantly inhibited the protein levels of FAS and VEGF in tumor-bearing mice (FIG. 17C). These in vivo results show that administration of HO-3867 inhibited tumor-growth through the inhibition of migration- and invasion-regulatory genes such as FAS and FAK in ovarian cancer xenografts in mice.

Discussion of Example 3

The results of the present Example 3 show for the first time that HO-3867 attenuates cancer cell migration and invasion through inhibition of FAS and FAK expression in ovarian cancer cell lines as well as in ovarian tumor xenografts in mice.

The results confirmed that FAS and FAK are indeed necessary for the motility of the ovarian cancer cells and that HO-3867 suppresses the levels of both FAS and FAK by promoting ubiquitination-dependent degradation process and inhibiting FAS-stabilizing genes of USP2a. HO-3867 also significantly inhibited the FAS activity, mRNA levels, and downstream proteins pERK1/2, pHER1, SREBP1, VEGF, and MMP-2. The Example 3 further confirmed the inhibition of FAS, FAK, and downstream proteins in tumor tissues obtained from A2780 xenograft tumor-bearing mice treated with HO-3867.

Thus, Example 3 shows that HO-3867 is capable of suppressing the migration and invasion of the ovarian cancer cells by inhibiting the expression/activity of FAS and FAK proteins.

Cell migration and invasion are vital elements involved in numerous physiological and pathological processes, including angiogenesis and metastasis. The high mortality rate among ovarian cancer patients is attributed not only to a lack of early detection and treatment, but also to the highly invasive (metastatic) nature of the disease. The poor prognosis associated with the treatment of ovarian cancer is mainly due to the late stage of disease with metastasis at presentation. Particularly, malignant ovarian surface epithelial cells primarily spread to adjacent organs by local invasion. The significant failure rate of chemotherapy in ovarian cancer patients with advanced stage of metastatic disease is also a main concern, suggesting cell motility as a potential therapeutic target for ovarian cancer treatment. In the present Example, 3, for the first time, we showed that HO-3867 acts as an effective blocker of ovarian cancer cell migration and invasion through inhibition of motility-promoting proteins including FAS, FAK, and VEGF.

Fatty acid synthase (FAS) is a metabolic enzyme involved in the synthesis of long-chain saturated fatty acids that are essential for membrane synthesis in proliferating cells. FAS is overexpressed in many human cancers including the carcinomas of the breast, prostate, stomach, lung, ovary and mesothelioma. The fact that overexpression of FAS is more pronounced in the clinically aggressive cancers suggest a functional role for FAS in the progression of malignant cancer. Inhibition of FAS activity preferentially attenuates tumor-cell growth by inducing apoptosis through inactivation of pAkt and dephosphorylation of Bad in ovarian cancer. Since FAS appears to provide a selective advantage to tumor progression, FAS has become a promising target for anticancer drug development. The present Example 3 shows that HO-3867 is capable of inhibiting both the expression and activity of FAS in A2780 and SKOV3 cells resulting in the attenuation of their ability to migrate and invade.

FAK is a focal adhesion-associated protein kinase involved in cellular adhesion and spreading processes. It serves as a key protein in the regulation of focal adhesion dynamics. FAK is a critical mediator of integrin adhesion turnover that promote cell migration. Like FAS, overexpression of FAK has also been found in most ovarian tumors, where it is shown to be associated with high aggressiveness and poor patient survival. Therefore, FAK is an attractive target for ovarian cancer therapeutics/preventions. In the present Example 3, we discovered that HO-3867 was capable of inhibiting FAK expression in the ovarian cancer cell lines tested.

Although the precise mechanism of FAS degradation have not yet been fully understood, it is evident that ubiquitination is involved in the pHO-3867-mediated proteasomal degradation of FAS. The isopeptidase USP2a is a preproteasomal, androgenregulated isopeptidase and is a key regulator of prostate cancer cell survival through the stabilization of FAS. FAS colocalizes and physically interacts with USP2a in cancer cells, suggesting that this isopeptidase rescues FAS from degradation and thereby prevents apoptosis. Of interest, the rapid downregulation in FAS was consistent with accelerated ubiquitin-dependent degradation. This effect was further confirmed by the observation that the proteasome inhibitor MG132 blocked the HO-3867-induced FAK degradation. We observed that HO-3867 significantly suppressed the FAS-regulating genes such as, pHER, SREBP1, pERK1/2, VEGF, and MMP-2 expression.

The present Example 3 provides the first evidence that HO-3867 inhibits the migration and invasion of ovarian cancer cells through downregulation of FAS and FAK. These results show that molecular targeting of FAS and FAK by HO-3867 is a useful strategy for ovarian cancer therapy.

### Example 4

### Pharmaceutical Compositions

The active ingredient(s)s of the invention, and derivatives, fragments, analogs, homologs pharmaceutically acceptable salts or hydrate thereof, can be incorporated into pharmaceutical compositions suitable for administration, together with a pharmaceutically acceptable carrier or excipient. Such compositions typically comprise a therapeutically effective amount of any of the active ingredient(s)s described herein, and a pharmaceutically acceptable carrier. Preferably, the effective amount is an amount effective to selectively induce terminal differentiation of suitable neoplastic cells and less than an amount which causes toxicity in a subject.

Any inert excipient that is commonly used as a carrier or diluent may be used in the active ingredient(s)s of the present invention, such as for example, a gum, a starch, a sugar, a cellulosic material, an acrylate, or mixtures thereof. The compositions may further comprise a disintegrating agent (e.g., croscarmellose sodium) and a lubricant (e.g., magnesium stearate), and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release active ingredient(s).

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active ingredient(s), use thereof in the compositions is contemplated. Supplementary active ingredient(s)s can also be incorporated into the compositions.

Non-limiting examples of solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Non-limiting examples of liquid active ingredient(s)s, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In certain embodiments, the active ingredient(s)s can be prepared with carriers that will protect the active ingredient(s) against rapid elimination from the body, such as a controlled release active ingredient(s), including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such active ingredient(s)s will be apparent to those skilled in the art. The materials can also be obtained commercially from Alza Corporation and Nova Pharmaceuticals, Inc. These can be prepared according to methods known to those skilled in the art.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active ingredient(s) calculated to produce the desired therapeutic effect in association with the required pharmaceutical carrier. The specification for the dosage unit forms of the invention are dictated by and directly dependent on the unique characteristics of the active ingredient(s) and the particular therapeutic effect to be achieved, and the limitations inherent in the art of compounding such an active ingredient(s) for the treatment of individuals.

The pharmaceutical compositions can be included in a container, pack, or dispenser together with instructions for administration. For example, the active ingredient(s)s may be administered intravenously on the first day of treatment, with oral administration on the second day and all consecutive days thereafter. The active ingredient(s)s of the present invention may be administered for the purpose of preventing disease progression or stabilizing tumor growth.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the active ingredient(s) administered to the subject is less than an amount that would cause toxicity in the subject. In the certain embodiments, the amount of the active ingredient(s) that is administered to the subject is less than the amount that causes a concentration of the active ingredient(s) in the subject to equal or exceed the toxic level of the active ingredient(s). The optimal amount of the active ingredient(s) that should be administered to the subject in the practice of the present invention will depend on the particular active ingredient(s) used and the type of cancer being treated.

The active ingredient(s) herein may also contain more than one active ingredient(s) (a second medicament), preferably those with complementary activities that do not adversely affect each other. The type and effective amounts of such medicaments depend, for example, on the amount and type of active ingredients present in the active ingredient(s), and clinical parameters of the subjects.

Kits

In a further aspect, there is provided herein a kit for use in treatment and prevention of a metabolic disorder, the kit comprising: i) individual dosage forms of a pharmaceutical composition according to the invention; and ii) instructions for administration of the pharmaceutical composition to a subject in need thereof.

In a further aspect, the invention provides a kit for use in treatment and prevention of a metabolic disorder, the kit comprising: i) individual dosage forms, and ii) instructions for administration of the dosage form to a subject in need thereof.

In Vitro Methods

The present invention also provides in-vitro methods for selectively inducing terminal differentiation, cell growth arrest and/or apoptosis of neoplastic cells thereby inhibiting proliferation of such cells, by contacting the cells with an effective amount of a composition containing ouabain, or a pharmaceutically acceptable salt or hydrate thereof.

Although the methods of the present invention can be practiced in vitro, it is contemplated that the preferred embodiment for the methods of selectively inducing terminal differentiation, cell growth arrest and/or apoptosis of neoplastic cells, and the like will comprise contacting the cells in vivo, i.e., by administering the compounds to a subject harboring neoplastic cells or tumor cells in need of treatment.

Treatments

The active ingredient(s) may be administered in any dose, provided it is effective to treat the patient. A physician having ordinary skill in the art can readily determine and prescribe the effective amount of the pharmaceutical composition required, depending on such factors as the particular active ingredient(s) employed, prior clinical experience, the patient's characteristics and clinical history, the type and severity of disease or disorder, other medicines being given, and any side effects predicted. For example, the physician could start with doses of an active ingredient(s), employed in the pharmaceutical composition at levels lower than that required in order to achieve the desired therapeutic effect and gradually increase the dosage until the desired effect is achieved. The effectiveness of a given dose or treatment regimen can be determined, for example, by assessing signs and symptoms and/or assessing inhibition of structural damage or of radiographic progression in the patient using the standard measures of efficacy.

The dose may be by weight or a fixed dose, preferably a fixed dose regardless of weight. An example of a weighted dose is 375 mg/m² weekly×4. As a general proposition, the effective amount of the antibody administered parenterally per dose will be in the range of about 20 mg to about 5000 mg, by one or more dosages, which can be translated to a dose by weight.

In an alternative aspect, one may administer a second medicament. The combined administration includes co-administration, using separate formulations or a single pharmaceutical formulation, and consecutive administration in either order, wherein preferably there is a time period while both (or all) active agents simultaneously exert their biological activities. The second medicament includes, for example, chemotherapeutic agents, immunosuppressive agents, antibodies, cytokine antagonists, integrin antagonist (e.g., antibody), corticosteroids, or any combination thereof.

Any of the compounds described above, including prodrugs and active compounds produced by the kit, can be combined with at least one pharmaceutically-acceptable carrier to produce a pharmaceutical composition. The pharmaceutical compositions can be prepared using techniques known in the art. The composition can be prepared by admixing the compound with a pharmaceutically-acceptable carrier. Many pharmaceutically-acceptable carriers are known to those skilled in the art. These most typically would be standard carriers for administration to humans, including solutions such as sterile water, saline, and buffered solutions at physiological pH, which may optionally contain certain pharmaceutically acceptable solvents such as ethanol or dimethylsulfoxide. Many pharmaceutically acceptable solid carriers are also well known to those of ordinary skill, such as for example many mono-, di-, and polysaccharides such as sucrose, lactose, starches, pectins, and the like, as well as semi-synthetic or synthetic polymer such as hydroxyalkyl celluloses, dextrans, polyacrylates, polyvinylpyrrolidones, and the like. The pharmaceutical carrier(s) must be “acceptable” in the sense of being compatible with the other ingredients of the composition and not overly deleterious to the recipient thereof.

The pharmaceutical compositions can include sterile aqueous or non-aqueous solutions, suspensions, and emulsions. Examples of aqueous or non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

The pharmaceutical compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

Pharmaceutical compositions suitable for oral administration can be presented as discrete unit dosage forms such as hard or soft gelatin capsules, cachets or tablets each containing a predetermined amount of the active ingredient; as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art, e.g., with enteric coatings.

Oral liquid preparations can be in the form of, for example, aqueous or oily suspensions, solutions, emulsions, syrups or elixirs, or can be presented as a dry product for constitution with water or other suitable vehicle before use. Such liquid preparations can contain conventional additives such as suspending agents, emulsifying agents, non-aqueous vehicles (which can include edible oils), or one or more preservative.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the compositions can be adapted to provide sustained release of the active ingredient employed, e.g., by combination thereof with certain hydrophilic polymer matrices, e.g., comprising natural gels, synthetic polymer gels or mixtures thereof. The pharmaceutical compositions according to the invention can also contain other adjuvants such as flavorings, coloring, antimicrobial agents, or preservatives.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

The publication and other material used herein to illuminate the invention or provide additional details respecting the practice of the invention, are incorporated by reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. An anti-cancer composition comprising a redox based curcumin derivative, DAP-F(p), having the structure:

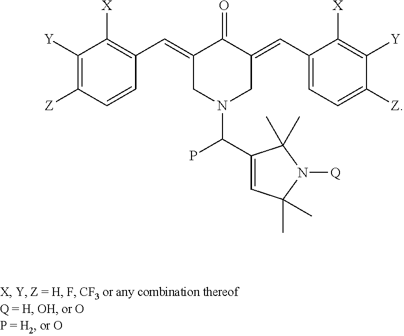

2. The composition of claim 1, comprising:

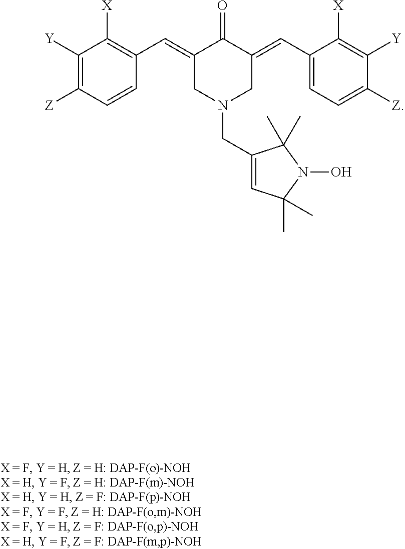

3. The composition of claim 1, comprising DAP-F(p)-NOH (1-[(1-oxyl-2,2,5,5-tetramethyl-2,5-dihydro-1H-pyrrol-3-yl)methyl]-(3E,5E)-3,5-bis(4-fluorobenzylidene)piperidin-4-one) [HO-3867]:

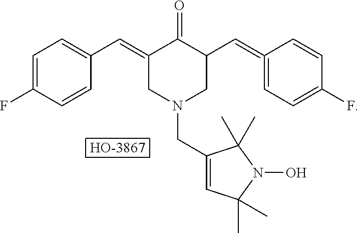

4. A method for providing protection to normal cells from associated oxidative damage in a subject in need thereof, while simultaneously providing a desired anti-cancer efficacy, comprising
administering to the subject an effective amount of at least one composition of claim 1, and pharmaceutically acceptable salts, solvates, isomers, tautomers, metabolites, analogs, pro-drugs, or derivatives thereof.

5. A method of treating and/or lessening the severity of a cancer in a subject in need thereof, comprising
administering to such subject a therapeutically effective amount of at least one composition of claim 1, and pharmaceutically acceptable salts, solvates, isomers, tautomers, metabolites, analogs, pro-drugs, or derivatives thereof.

6. A method for inducing a reduction of cell viability in cancer cells in a subject in need thereof, while having little adverse effect on viability of normal cells, comprising:
administering to the subject an effective amount of at least one composition of claim 1, and pharmaceutically acceptable salts, solvates, isomers, tautomers, metabolites, analogs, pro-drugs, or derivatives thereof.

7. A method for inducing a dose dependent generation of ROS in cancer cells and no significant induction of ROS in normal cells in a subject in need thereof, comprising:
administering to a subject in need thereof an effective amount of at least one composition of claim 1, and pharmaceutically acceptable salts, solvates, isomers, tautomers, metabolites, analogs, pro-drugs, or derivatives thereof.

8. A method for inducing G₂/M cell cycle arrest in cancer cells, comprising:
administering to the subject an effective amount of at least one composition of claim 1, and pharmaceutically acceptable salts, solvates, isomers, tautomers, metabolites, analogs, pro-drugs, or derivatives thereof.

9. A method for treating a disease or a disorder, comprising administering to a subject in need thereof an effective amount of at least one composition of claim 1, and pharmaceutically acceptable salts, solvates, isomers, tautomers, metabolites, analogs, pro-drugs, or derivatives thereof.

10. The method of claim 9, wherein the disease or the disorder is cancer.

11. The method of claim 9, wherein the cancer is one or more of: ovarian cancers, breast cancers, colon cancers, head and neck cancers; liver cancers; lung cancers, and prostate cancers.

12. The method of claim 9, wherein cancer is an ovarian cancer.

13. A method of inducing apoptosis in a cell in a subject in need thereof, comprising administering to a subject in need thereof an effective amount of at least one composition of claim 1, and pharmaceutically acceptable salts, solvates, isomers, tautomers, metabolites, analogs, pro-drugs, or derivatives thereof, thereby inducing apoptosis in the cell.

14. The method of claim 13, wherein the cell is a cancer cell.

15. The method of claim 4, wherein the subject is a human.

16. The method of claim 4, or wherein the cancer is an ovarian cancer.

17. A method of inhibiting cancer cell growth by promoting cell apoptosis, the method comprising the step of contacting the cancer cell with an agent that:
i) inhibits the function or expression of JAK/STAT3 signaling pathway,
ii) induces G₂-M cell cycle arrest by modulating cell cycle regulatory molecules p53, p21, p27, cyclin-dependent kinase 2, and cyclin;
iii) promotes apoptosis by caspase-8 and caspase-3 activation;
iv) induces cleavage of caspase-3, caspase-7, PARP as well as caspase-8, as well as inducing an increase in as/CD95 expression, decreasing the expression level of Ser727 p-Stat3 and Tyr705 p-Stat3, while maintaining the total Stat3 level,
v) induces a dose dependent generation of ROS in cancer cells and no significant induction of ROS in normal cells
vi) causes an increase in the expression of functional Fas/DC95;
v) suppresses migration and invention of cancer cells by inhibiting expression/activity of fatty acid synthase (FA) and focal adhesion kinase (FAK) proteins; and/or
vi) causes decrease in signal transducers and activator of transcription (STAT3, Try705, Ser727) and JAK1 phosphorylation, thereby inhibiting cancer cell growth.

18. The method of claim 17, wherein the agent comprises at least one composition of comprising a redox based curcumin derivative, DAP-F(p), having the structure:

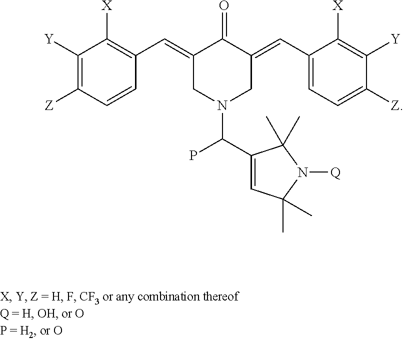

19. The method of claim 17, wherein the agent is HO-3867 (1-[(1-oxyl-2,2,5,5-tetramethyl-2,5-dihydro-1H-pyrrol-3-yl)methyl]-(3E,5E)-3,5-bis(4-fluorobenzylidene)piperidin-4-one).

20. A method for regulating, in a subject in need thereof, the down-regulation of suppressors of apoptosis, comprising:
administering to the subject an effective amount of at least one composition of claim 1, and pharmaceutically acceptable salts, solvates, isomers, tautomers, metabolites, analogs, pro-drugs, or derivatives thereof.

21. A method of treating an acute or a chronic free-radical associated disease in a subject in need thereof, the method comprising:
administering to the subject an effective amount of at least one composition of claim 1, and pharmaceutically acceptable salts, solvates, isomers, tautomers, metabolites, analogs, pro-drugs, or derivatives thereof.

22. The method of claim 20, wherein the agent is HO-3867 (1-[(1-oxyl-2,2,5,5-tetramethyl-2,5-dihydro-1H-pyrrol-3-yl)methyl]-(3E,5E)-3,5-bis(4-fluorobenzylidene)piperidin-4-one).
